(19) United States
(12) Patent Application Publication
(10) Pub. No.: US 2009/0202620 A1
(43) Pub. Date: Aug. 13, 2009

(54) POLYMER-STABILIZED LIPOSOMAL COMPOSITIONS AND METHODS OF USE

(75) Inventors: William G. Turnell, San Diego, CA (US); Geoffrey C. Landis, Carlsbad, CA (US); Benjamin W. Parcher, San Diego, CA (US); Alan Dominguez, Porter Ranch, CA (US)

Correspondence Address: DLA PIPER LLP (US), 4365 EXECUTIVE DRIVE, SUITE 1100, SAN DIEGO, CA, 92121-2133, US

(73) Assignee: MediVas, LLC, San Diego, CA (US)

(21) Appl. No.: 11/849,989

(22) Filed: Sep. 4, 2007

Related U.S. Application Data
(60) Provisional application No. 60/842,422, filed on Sep. 5, 2006.

(51) Int. Cl. A61K 9/127; A61K 39/00; A61K 38/02; A61K 38/28

(52) U.S. Cl. 424/450; 424/184.1; 514/2; 514/3

(57) ABSTRACT

The invention provides liposomal particle compositions, which incorporate a lipophilic biodegradable polymer, such as amino acid-containing polyester amide (PEA), polyester urethane (PEUR), and polyester urea (PEU), throughout the particles to stabilize the composition for in vivo delivery in of an incorporated biologic agent. For oral delivery, a biologic, such as insulin, is conjugated directly to the polymer. Lipids in the particle are selected to further stabilize the composition during fabrication and digestion, providing sustained delivery of the biologic with native activity. Methods of making and using the invention compositions to administer the biologic agent in vivo are also included.

### RELATED APPLICATIONS

This application relies for priority under 35 U.S.C. § 119(e) on U.S. Ser. No. 60/842,422, filed Sep. 5, 2006, and claims the benefit under 35 U.S.C. § 120 of U.S. Ser. No. 11/344,689, filed Jan. 31, 2006 and U.S. Ser. No. 11/603,660, filed Nov. 21, 2006, each of which is hereby incorporated by reference in its entirety.

### 1. FIELD OF THE INVENTION

The invention relates generally to the field of liposome technology and pharmacotherapy, and, more specifically, to polymer-stabilized liposomal formulations and to methods for stabilizing liposomal formulations for use in pharmacotherapy.

### 2. DESCRIPTION OF THE RELATED ART

Liposomes are artificial lipid or phospholipid vesicles enclosing aqueous internal chambers into which bioactive agents, such as drugs and biologics, can be entrapped with the intention of achieving controlled release of the bioactive agent after administration of the liposomal particle to an individual.

Due to its polar nature, the head of a phospholipid is hydrophilic, while the nonpolar tails are hydrophobic. When placed in water, phospholipids form a bilayer, known as a “lipid bilayer,” where the hydrophobic tails line up against each other, forming a lipid bilayer with hydrophilic heads on both sides extending out into the water. (This behavior allows the phospholipids to spontaneously form vesicles or liposomes). Due to these electrical properties, water-soluble bioactive agents added to the water are entrapped inside the aggregation of the hydrophobic ends and fat-soluble bioactive agents are entrapped into a lipid bilayer in the vesicles or liposomes.

Several types of liposomes have been described (U.S. Pat. No. 4,552,803 to Lenk; U.S. Pat. No. 4,310,506 to Baldeschwieler; U.S. Pat. No. 4,235,871 to Papahadjopoulos; U.S. Pat. No. 4,224,179 to Schneider; U.S. Pat. No. 4,078,052 to Papahadjopoulos; U.S. Pat. No. 4,394,372 to Taylor; U.S. Pat. No. 4,308,166 to Marchetti; U.S. Pat. No. 4,485,054 to Mezei; and U.S. Pat. No. 4,508,703 to Redziniak; Szoka, et al. (1980) Ann. Rev. Biophys. Bioeng. 9:465-508; Liposomal particles, Marc J. Ostro, Ed., Marcel-Dekker, Inc., New York, 1983; Poznansky and Juliano, Pharmacol. Rev. (1984) 36:277-236; Kim, et al. Biochim. Biophys. Acta (1983) 728:339-348; Kim et al. Biochim. Biophys. Acta (1981) 646:1-10).

Unilamellar liposomes have a single bilayer membrane enclosing an aqueous volume (Huang, 1969, Biochemistry (1969) 8:334-352) while multilamellar liposomes have multiple concentric membranes arranged in an “onion-skin” structure, in between which are shell-like concentric aqueous compartments (Bangham et al. J. Mol. Biol. (1965) 13:238-252). Multivesicular liposomes are microscopic lipid-vesicles consisting of intersecting lipid bilayer membranes, enclosing multiple non-concentric aqueous chambers and characterized by a neutral lipid separating the leaflets of a bilayer membrane. (Kim et al. Biochim. Biophys. Acta (1983) 728:339-348).

Liposomes have been described for delivery of many molecules (e.g., insulin and some cancer drugs) to the body and for treating diseases that affect the phagocytes of the immune system because liposomes tend to accumulate in the phagocytes, which recognize them as foreign invaders. For this reason liposomes have also been described as useful in vaccines (Reddy, R. et al. J. Immunol. (1992) 148:1585 and Rock, K. L., Immunol. Today (1996) 17:131).

However, in use studies of liposomes have shown a discouraging difference in drug retention time in saline and in serum, due to premature release of the active agent. For example, liposomes encapsulating Vincristine, a chemotherapy agent, have shown a retention time of more than eight weeks in saline, but less than one hour in serum (Webb et al., Cancer Chemotherapy and Pharmacology 42:461 (1998)). In another study, liposomes encapsulating Ciprofloxacin, an antibiotic, demonstrated a retention time of more than four weeks in saline, but less then 30 minutes in serum (Bakker-Woudenberg et al., Antimicrobial Agents (2001) 45:1487).

To overcome the problem of premature release of the active agent upon systemic administration, various methods and compounds have been used for stabilizing liposomes. Among the compounds added to liposome formulations to increase residence time in the blood are short chain fatty acids (U.S. Pat. No. 5,653,998), stabilizers including sterols, such as cholesterol, which reduce membrane fluidity; glycerol, or saccharides, such as sucrose; lipid surfactants, such as lysolipids (U.S. Pat. No. 5,882,679); and hydrophilic polymers, such as polyethylene glycol, dextran, pullulan, Ficoll, polyvinyl alcohol synthetic polyamino acid, amylose, amylopectin, chitosan, mannan, cyclodextrin, pectin, and carrageenan (U.S. Pat. Nos. 6,228,391 and 6,660,525).

Despite the many developments in the art designed to prevent premature release of the active agent from liposomes upon systemic administration, there is a need for new and better liposomal compositions and methods for stabilizing the release rate of bioactive agents from such liposomal compositions.

Unlike organic or inorganic drugs and small biologics, such as short peptides, the function of biologic macromolecules, and in particular of proteins, depends upon the constancy of the three-dimensional structure of the molecule. The spatial, conformational fold of the macromolecular chain is held together by the concerted action of forces, each of which is far weaker than the covalent bonds of the macromolecular chain itself. All of these non-covalent forces are fundamentally electronic in nature: electrostatic ionic forces (including hydrogen bonding) or electrodynamic dispersion forces (short range hydrophobicity).

Open formulations, such as hydrogels, work to preserve therapeutic function by allowing the biologic molecules to bathe in the natural aqueous milieu. Extensive direct and water-bridged hydrogen bonding between the gel polymer and the biologic, in some cases coupled with local hydrophobic interactions, limit release of the biologic by diffusion through the gel. However, in many cases such open formulations allow ingress of degrading enzymes, which can infiltrate through the similarly sized pores of the gel, presenting an inherent problem for the delivery of structurally intact biologic macromolecules.

Greater protection has been provided to a macromolecular biologic by hydrophobic polymers, which present a denser structure for the matrixing or encapsulation of a macromolecular biologic. However, as hydrophobic polymers repel water, such synthetic polymer formulations have heretofore demonstrated limited capacity for molecular interactions that help to preserve the active (e.g., native), folded state of the molecule. For example, the hydrophobic polyesters (e.g. PLGA) possess only limited ionic bonding capacity. In particular, polyesters lack hydrogen bond donors. Similarly, methacrylates are hydrophobic and must be extensively derivatized to introduce other, non-covalent bonding capacities. Moreover, most synthetic hydrophobic polymers have poor bio-erosion properties, or degrade via water/acid hydrolysis, resulting in degradation products that can modify the macromolecular biologic whose protection is being sought.

Chemists, biochemists, and chemical engineers are all looking beyond traditional polymer networks to find other innovative drug transport systems. Thus, there is also still a need in the art for new and better polymer compositions for controlled delivery of a variety of different types of bioactive agents, including macromolecular biologics.

### SUMMARY OF THE INVENTION

The invention is based on the discovery that polymers with lipophilic properties, especially those that are capable of hydrogen bonding to discrete water molecules, such as those that incorporate whole amino acids, can be used to stabilize liposomal particles. It has been discovered that the stabilizing polymers with lipophilic properties in the liposomal particles of the invention compositions permeate the entire particles and interact with lipid and lipid-acting components of the particles. Therefore, unlike true liposomes, the particles of the invention polymer-stabilized liposomal compositions lack separate lipid and aqueous compartments and can be lyophilized without substantial loss of activity of a sequestered bioactive agent.

The stabilizing polymers in the invention compositions also interact with bioactive agents entrapped therein to facilitate release of the bioactive agent in a consistent and controlled manner when administered in vivo, for example, systemically. Interactions of the stabilizing polymers with other components in the liposomal particles not only provide an integrating and stabilizing physical structure during particle fabrication, but also continue to stabilize and maintain the active structure of a sequestered bioactive agent, e.g., as a “folded” protein, when the composition has been administered in vivo.

Accordingly, in one embodiment, the invention provides a polymer-stabilized liposomal composition in which liposomal particles containing a stabilizing polymer with lipophilic properties integrally located throughout the particles and at least one vesicle-forming lipid or lipid-acting compound sequester at least one bioactive agent so as to maintain the substantial native activity thereof. The term “liposomal” as used throughout the specification and claims herein to describe the invention compositions, including the particles thereof, means that the particles share certain characteristics of liposomes, but are not true liposomes, because the particles of the invention compositions lack the separate lipid and aqueous compartments that characterize true liposomes.

In another embodiment, the invention provides methods for delivering a bioactive agent to a subject by administering in vivo to the subject an invention polymer-stabilized liposomal composition so that the composition biodegrades to release a sequestered bioactive agent with substantial native activity to the subject in vivo at a controlled rate.

In yet another embodiment, the invention provides methods for making a polymer-stabilized liposomal composition, by combining the following components in a first liquid so as to form a first homogeneous liquid solution:

a) a bioactive agent having a native activity;

b) a stabilizing polymer with lipophilic properties; and

c) at least one lipid or lipid-acting compound selected from:
Class I, non-swelling and non-polar organic-based stabilizers;
Class II, organic based surfactants that are swelling amphiphiles; and
Class IV, cholesterol or cholesterol-based compounds.
   The first homogeneous liquid solution is then emulsified in a second liquid in which the components are not soluble so as to obtain an emulsion of droplets of the first homogeneous liquid solution in the second liquid. The first liquid is evaporated from the emulsion so as to yield stable liposomal particles comprising the components a), b) and c) with the bioactive agent entrapped in concert by the stabilizing polymer and the at least one lipid or lipid-acting compound so as to retain substantial native activity.

### A BRIEF DESCRIPTION OF THE FIGURES

FIG. 1 is a schematic drawing illustrating stabilization of free insulin by a minority of insulin protomers conjugated to a PEA polymer of structural formula (IV).

FIG. 2 is a key to the components of the invention composition for oral delivery of a biologic.

FIG. 3 is a pie chart showing the relative proportions (wt %) of the components of an invention composition with segments shaded as shown in FIG. 2.

FIG. 4 is a schematic representation illustrating biological transformation of the polymer stabilized liposomal micro-particles from FIG. 3 to nano-particles by the action of digestive enzymes. Relative proportions of the components of the particles are shaded as in FIGS. 2 and 3.

FIGS. 5 A-B are graphs showing the bioactivity and bioavailability of insulin delivered orally in particles of an invention composition. Enteric coated gelatin capsules containing the formulation were administered orally to fasted rats, and blood glucose and insulin levels were measured over 5 hours.

FIG. 6 is a graph showing the bioactivity of insulin delivered orally in particles of the invention composition. Solutions containing the particle were administered to rats by oral gavage, and blood glucose levels were measured over 5 hours. Three different doses were tested and a dose response was observed in which 30 and 60 IU insulin/kg of rat body weight resulted in a depression of glucose levels. Test glucose levels are presented as a percentage of the time zero value for each dose.

FIG. 7 is a graph showing the glucose levels over time in mice orally administered particles of invention polymer-stabilized liposomal composition containing human insulin 15 minutes before an oral glucose challenge of 1.5 g/kg. (▪)=control mice (no formulation); (♦)=mice orally administered 10 IU/kg insulin formulated in particles of invention composition.

FIG. 8 is a graph showing the glucose levels over time in mice orally administered enteric coated particles of an invention polymer-stabilized liposomal composition containing human insulin 15 minutes before an oral glucose challenge of 1.5 g/kg. (▪)=control mice (no formulation); (♦)=mice orally administered 10 IU/kg insulin formulated in invention enteric coated particles.

### DETAILED DESCRIPTION OF THE INVENTION

The invention is based on the discovery that biodegradable polymers that have lipophilic segments and preferably also can hydrogen bond to water molecules, such as the poly(ester amide) (PEA), poly(ester urethane) (PEUR) and poly(ester urea) (PEU) polymers disclosed herein, stabilize liposomal particle compositions for release of incorporated bioactive agents in a consistent and controlled manner when administered in vivo. The bio-analogous PEAs, PEURs and PEUs used in the invention compositions and methods comprise nontoxic building blocks, such as hydrophobic α-amino acids as well as aliphatic diols and di-carboxylic acids containing hydrocarbon segments, which render these stabilizing polymers lipophilic. At the same time, the polymers control the release rate of sequestered bioactive agents from the invention polymer-stabilized liposomal composition by degrading at an enzyme-controlled rate.

Accordingly, in one embodiment, the invention provides a polymer-stabilized liposomal composition comprising a liposomal particle having at least one lipid bilayer, at least one bioactive agent incorporated by the liposomal particle, and a biodegradable, stabilizing polymer with lipophilic properties included in the lipid bilayer of the liposomal particle, wherein the bioactive agent is attached directly to the stabilizing polymer via a functional group thereof. In one embodiment, the stabilizing polymer comprises at least one or a blend of the PEA, PEUR and PEU polymers described herein.

The Biodegradable Stabilizing Polymer with Lipophilic Properties

Stabilizing polymers useful in the invention polymer-stabilized liposomal compositions have lipophilic properties, at least in segments therein, and preferably hydrogen bond to water molecules. These stabilizing polymers additionally may feature free functional groups to which a bioactive agent can be attached, for example, covalently. Many biodegradable and/or biocompatible lipophilic polymers known in the art can be used in manufacture of the invention polymer-stabilized liposomal composition, including the following: Aliphatic polyesters, such as polylactic acid, polyglycolic acid and their copolymers; Poly(ε-caprolactone), poly(δ-valerolactone), polyesters with longer (i.e., C₁₅ to C₂₅) hydrocarbon chains; Dendritic polymers of polyesters containing a modified terminal hydroxyl; Aliphatic and aromatic polycarbonates (PC); Aliphatic polyamides, polypeptides (such as diblock-copolypeptides, which are polymeric amphiphiles containing both water soluble and insoluble domains); Polyesteramides; Polyurethanes; Silicones, such as poly(dimethylsyloxanes); Lipophilic cyclo- and poly(phosphazenes); Poly(methacrylic acid), poly(styrene) and hydrophobic polyacrylic, polyvinyl and polystyrene carriers.

The PEA, PEUR and PEUR polymers described structurally herein contain lipophilic properties (i.e. lipophilic segments) sufficient for the stabilizing polymer to be incorporated into the lipids of liposomal particles, hydrophobic segments for binding to the hydrophobic regions of the liposomal particles, and hydrophilic segments for stabilizing the polar regions of the liposomal particles. In addition these PEA, PEUR and PEUR polymers also have free functional groups therein suitable for attachment of bioactive agents to the stabilizing polymer. During use for delivery of a bioactive agent to tissues of a subject, the lipophilic segments of the stabilizing polymers in the invention compositions also bind to cell lipid membranes to aid in delivery of the bioactive agent to the tissues.

Therefore, in one embodiment, the stabilizing polymer in the invention polymer-stabilized liposomal compositions comprises at least one or a blend of the following: a PEA having a chemical formula described by structural formula (I),

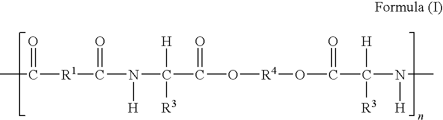

wherein n ranges from about 5 to about 150; R¹ is independently selected from (C₂-C₂₀) alkylene, (C₂-C₂₀) alkenylene, α,ω-bis(4-carboxyphenoxy) (C₁-C₈) alkane, residues of 3,3′-(alkanedioyldioxy) dicinnamic acid or 4,4′-(alkanedioyldioxy) dicinnamic acid, residues of α,ω-alkylene dicarboxylates of formula (III), and combinations thereof; wherein R⁵ and R⁶ in Formula (III) are independently selected from (C₂-C₁₂) alkylene or (C₂-C₁₂) alkenylene; the R³s in individual n monomers are independently selected from the group consisting of hydrogen, (C₁-C₆) alkyl, (C₂-C₆) alkenyl, (C₆-C₁₀) aryl (C₁-C₆) alkyl and —(CH₂)₂SCH₃; and R⁴ is independently selected from the group consisting of (C₂-C₂₀) alkylene, (C₂-C₂₀) alkenylene, (C₂-C₈) alkyloxy (C₂-C₂₀) alkylene, bicyclic-fragments of 1,4:3,6-dianhydrohexitols of structural formula (II), and combinations thereof;

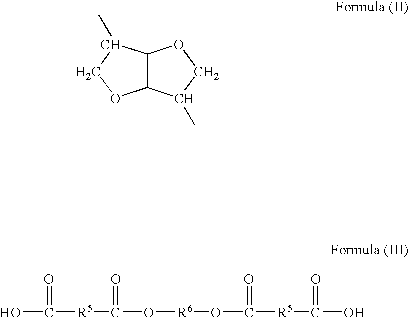

or a PEA having a chemical formula described by structural formula (IV):

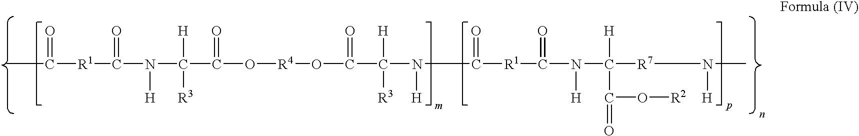

wherein n ranges from about 5 to about 150, m ranges about 0.1 to 0.9: p ranges from about 0.9 to 0.1; wherein R¹ is independently selected from (C₂-C₂₀) alkylene, (C₂-C₂₀) alkenylene, α,ω-bis(4-carboxyphenoxy) (C₁-C₈) alkane, residues of 3,3′-(alkanedioyldioxy) dicinnamic acid or 4,4′-(alkanedioyldioxy) dicinnamic acid, residues of α,ω-alkylene dicarboxylates of formula (III), and combinations thereof; wherein R⁵ and R⁶ in Formula (III) are independently selected from (C₂-C₁₂) alkylene or (C₂-C₁₂) alkenylene; each R² is independently hydrogen, (C₁-C₁₂) alkyl, (C₂-C₈) alkyloxy, (C₂-C₂₀) alkyl (C₆-C₁₀) aryl or a protecting group; the R³s in individual m monomers are independently selected from the group consisting of hydrogen, (C₁-C₆) alkyl, (C₂-C₆) alkenyl, (C₆-C₁₀) aryl (C₁-C₆) alkyl and —(CH₂)₂SCH₃; and R⁴ is independently selected from the group consisting of (C₂-C₂₀) alkylene, (C₂-C₂₀) alkenylene, (C₂-C₈) alkyloxy (C₂-C₂₀) alkylene, bicyclic-fragments of 1,4:3,6-dianhydrohexitols of structural formula (II), and combinations thereof, and R⁷ is independently (C₁-C₂₀) alkyl or (C₂-C₂₀) alkenyl, for example, (C₃-C₆) alkyl or (C₃-C₆) alkenyl;

a PEUR) having a chemical formula described by structural formula (V),

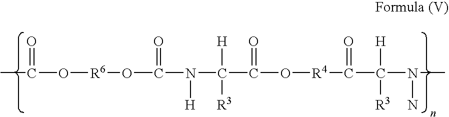

and wherein n ranges from about 5 to about 150; wherein the R³s within an individual n monomer are independently selected from the group consisting of hydrogen, (C₁-C₆) alkyl, (C₂-C₆) alkenyl, (C₆-C₁₀) aryl(C₁-C₆) alkyl and —(CH₂)₂SCH₃; R⁴ and R⁶ is selected from the group consisting of (C₂-C₂₀) alkylene, (C₂-C₂₀) alkenylene, (C₂-C₈) alkyloxy (C₂-C₂₀) alkylene, bicyclic-fragments of 1,4:3,6-dianhydrohexitols of structural formula (II), and combinations thereof;

or a PEUR having a chemical structure described by general structural formula (VI),

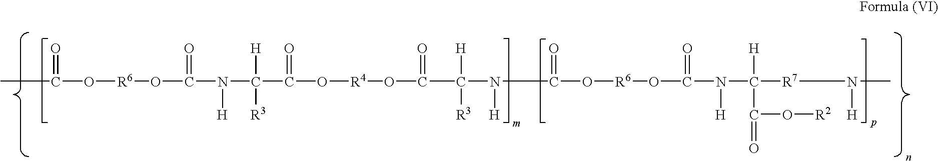

wherein n ranges from about 5 to about 150, m ranges about 0.1 to about 0.9: p ranges from about 0.9 to about 0.1; R² is independently hydrogen, (C₁-C₁₂) alkyl, (C₂-C₈) alkyloxy (C₂-C₂₀) alkyl, (C₆-C₁₀) aryl or a protecting group; the R³s within an individual m monomer are independently selected from the group consisting of hydrogen, (C₁-C₆) alkyl, (C₂-C₆) alkenyl, (C₆-C₁₀) aryl (C₁-C₆) alkyl and —(CH₂)₂SCH₃; R⁴ and R⁶ is independently selected from (C₂-C₂₀) alkylene, (C₂-C₂₀) alkenylene, (C₂-C₈) alkyloxy (C₂-C₂₀) alkylene, bicyclic-fragments of 1,4:3,6-dianhydrohexitols of structural formula (II), and combinations thereof, and R⁷ is independently (C₁-C₂₀) alkyl or (C₂-C₂₀) alkenyl, for example, (C₃-C₆) alkyl or (C₃-C₆) alkenyl;

or a PEU having a chemical formula described by structural formula (VII),

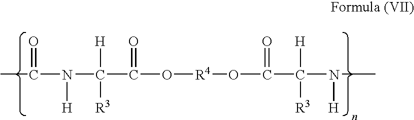

wherein n is about 10 to about 150; the R³s within an individual n monomer are independently selected from hydrogen, (C₁-C₆) alkyl, (C₂-C₆) alkenyl, (C₆-C₁₀) aryl (C₁-C₆)alkyl and —(CH₂)₂SCH₃; R⁴ is independently selected from (C₂-C₂₀) alkylene, (C₂-C₂₀) alkenylene, (C₂-C₈) alkyloxy (C₂-C₂₀) alkylene; or a bicyclic-fragment of a 1,4:3,6-dianhydrohexitol of structural formula (II) and combinations thereof;

or a PEU having a chemical formula described by structural formula (VIII),

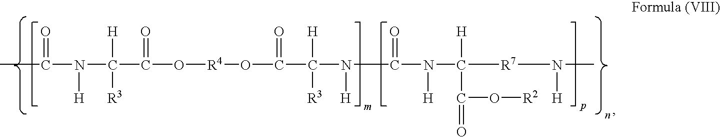

wherein m is about 0.1 to about 1.0; p is about 0.9 to about 0.1; n is about 10 to about 150; each R² is independently hydrogen, (C₁-C₁₂) alkyl, (C₂-C₈) alkyloxy (C₂-C₂₀) alkyl, (C₆-C₁₀) aryl or a protecting group; the R³s within an individual m monomer are independently selected from hydrogen, (C₁-C₆) alkyl, (C₂-C₆) alkenyl, (C₆-C₁₀) aryl (C₁-C₆)alkyl and —(CH₂)₂SCH₃; R⁴ is independently selected from (C₂-C₂₀) alkylene, (C₂-C₂₀) alkenylene, (C₂-C₈) alkyloxy (C₂-C₂₀) alkylene; or a bicyclic-fragment of a 1,4:3,6-dianhydrohexitol of structural formula (II), or a combination thereof, and R⁷ is independently (C₁-C₂₀) alkyl or (C₂-C₂₀) alkenyl, for example, (C₃-C₆) alkyl or (C₃-C₆) alkenyl.

For example in one embodiment of the PEA polymer, at least one R¹ is a (C₂-C₂₀) alkylene or residue of α,ω-bis(4-carboxyphenoxy) (C₁-C₈) alkane and R⁴ is a bicyclic-fragment of a 1,4:3,6-dianhydrohexitol of general formula (II). In another alternative, R¹ in the PEA polymer is either a residue of α,ω-bis(4-carboxyphenoxy) (C₁-C₈) alkane, or a residue of 4,4′-(alkanedioyl dioxy) dicinnamic acid, or a combination thereof. In yet another alternative in the PEA polymer, R¹ is a residue α,ω-bis(4-carboxyphenoxy) (C₁-C₈) alkane, such as 1,3-bis(4-carboxyphenoxy)propane (CPP), or 4,4′-(adipoyldioxy) dicinnamic acid, and R⁴ is a bicyclic-fragment of a 1,4:3,6-dianhydrohexitol of general formula (II), such as 1,4:3,6-dianhydrosorbitol (DAS).

In one alternative in the PEUR polymer, at least one of R⁴ or R⁶ is a bicyclic fragment of 1,4:3,6-dianhydrohexitol, such as 1,4:3,6-dianhydrosorbitol (DAS).

In another alternative in the PEU polymer, at least one R⁴ is a bicyclic fragment of a 1,4:3,6-dianhydrohexitol, such as DAS.

For strongly hydrophobic segments in PEA, PEUR or PEU polymers, components such as di- L-leucine ester of 1,4:3,6-dianhydro-D-sorbitol or rigid aromatic di-acid like α,ω-bis(4-carboxyphenoxy) (C₁-C₈) alkane may be included in the polymer repeat unit.

As used herein, to describe the PEA, PEUR and PEU polymers of structural formulas (I and IV-VIII), the terms “amino acid” and “α-amino acid” mean a chemical compound containing an amino group, a carboxyl group and a R group, such as the R³ and R⁵ groups defined herein. As used herein, the term “biological α-amino acid” means the amino acid(s) used in synthesis are selected from phenylalanine, leucine, glycine, alanine, valine, isoleucine, methionine, or a mixture thereof. Additional biological amino acids used in fabrication of co-polymers include lysine and ornithine, but which are oriented in the polymer backbone adirectionally (i.e., in a non-biological orientation) such that the carboxyl group of the amino acid is pendent rather than being incorporated into a peptide bond. The biodegradable PEA, PEUR and PEU polymers useful in forming the invention compositions may contain multiple different α-amino acids in a single polymer molecule, for example, at least two different amino acids per repeat unit, or a single polymer molecule may contain multiple different amino acids in the polymer molecule, depending upon the size of the molecule.

The terms, “biodegradable” and “biocompatible” as used herein to describe the stabilizing PEA, PEUR and PEU polymers, including mixtures and blends thereof, means the polymer is capable of being broken down into innocuous products in the normal functioning of the body by water and/or by enzymes found in tissues of mammalian subjects, such as humans, and a variety of other mammalian subjects, such as pets (for example, cats, dogs, rabbits, ferrets), farm animals (for example, swine, horses, mules, dairy and meat cattle) and race horses when used as described herein.

The term “lipophilic” as used herein to describe certain properties of the stabilizing polymers useful in fabrication of the invention compositions means that the polymer is both hydrophobic or has hydrophobic segments therein and is capable of polar interactions.

The term “controlled” as used herein to described the release of bioactive agent(s) from invention polymer-stabilized liposomal compositions means the stabilizing polymer degrades over a desired period of time to provide a smooth and regular (i.e. “controlled”) time release profile of the sequestered bioactive agent (e.g., avoiding an initial irregular spike in drug release and providing instead a substantially smooth rate of change of release throughout biodegradation of the invention composition). The biodegradable polymers used in the invention polymer-stabilized liposomal composition can be designed to tailor the rate of biodegradation of the stabilizing polymer to result in release of a bioactive agent sequestered in the liposomal particles over a selected period of time. The stabilizing PEA, PEUR or PEU polymer, described herein by formulas (I and IV-VIII), when used in vivo will biodegrade over a time range selected from about 5 to 12 hours to about 8 hours to 5 months; preferably, about 5 to 8 hours to about 12 to 36 hours; for example, from about 12 hours to about 36 hours, or from about 8 hours to about 12 hours. Longer time spans within these ranges are particularly suitable for providing a liposomal composition that eliminates the need to repeatedly inject the composition to obtain a suitable therapeutic, diagnostic or palliative result.

The PEUs, PEURs and PEUs described herein as suitable for use as the stabilizing polymer of the invention compositions include hydrolyzable ester and enzymatically cleavable amide linkages that provide biodegradability, and are typically chain terminated, predominantly with amino groups. Optionally, the amino termini of the polymers can be acetylated or otherwise capped by conjugation to any other acid-containing, biocompatible molecule, to include without restriction organic acids, bioinactive biologics, and bioactive agents as described herein.

In one alternative, at least one of the α-amino acids used in fabrication of the stabilizing polymers used in the invention compositions and methods is a biological α-amino acid. For example, when the R³s are CH₂Ph, the biological α-amino acid used in synthesis is L-phenylalanine. In alternatives wherein the R³s are CH₂—CH(CH₃)₂, the polymer contains the biological α-amino acid, L-leucine. By varying the R³s within monomers as described herein, other biological α-amino acids can also be used, e.g., glycine (when the R³s are H), alanine (when the R³s are CH₃), valine (when the R³s are CH(CH₃)₂), isoleucine (when the R³s are CH(CH₃)—CH₂—CH₃), phenylalanine (when the R³s are CH₂—C₆H₅), or methionine (when the R³s are —(CH₂)₂SCH₃, and mixtures thereof. In yet another alternative embodiment, all of the various α-amino acids contained in the stabilizing polymers used in making the invention polymer-stabilized liposomal compositions are biological α-amino acids, as described herein. Biocompatibility of the stabilizing polymers is especially true when the amino acids used in fabrication of the polymers are biological L-α-amino acids.

Suitable protecting groups for use in the PEA, PEUR and PEU polymers include t-butyl or another as is known in the art. Suitable 1,4:3,6-dianhydrohexitols of general formula (II) include those derived from sugar alcohols, such as D-glucitol, D-mannitol, or L-iditol. Dianhydrosorbitol is the presently preferred bicyclic fragment of a 1,4:3,6-dianhydrohexitol for use in the PEA, PEUR and PEU polymers used in fabrication of the invention compositions.

The term “aryl” is used with reference to structural formulae herein to denote a phenyl radical or an ortho-fused bicyclic carbocyclic radical having about nine to ten ring atoms in which at least one ring is aromatic. In certain embodiments, one or more of the ring atoms can be substituted with one or more of nitro, cyano, halo, trifluoromethyl, or trifluoromethoxy. Examples of aryl include, but are not limited to, phenyl, naphthyl, and nitrophenyl.

The term “alkenylene” is used with reference to structural formulae herein to mean a divalent branched or unbranched hydrocarbon chain containing at least one unsaturated bond in the main chain or in a side chain. The polymers disclosed herein (e.g., those having structural formulas (I and IV-VIII), upon enzymatic degradation, provide biological or non biological amino acids, while the other breakdown products can be metabolized in biochemical pathways equivalent to those for fatty acids and sugars. Uptake of the polymer in vivo is safe: studies have shown that a subject can metabolize/clear the polymer degradation products. These polymers and the invention polymer-stabilized liposomal compositions utilizing such polymers are, therefore, substantially non-inflammatory to the subject both at the site of administration, apart from the trauma caused by injection itself, and systemically, and are particularly suited or oral or intra-nasal delivery.

The molecular weights and polydispersities herein are determined by gel permeation chromatography (GPC) using polystyrene standards. More particularly, number and weight average molecular weights (Mₙ and M_{w}) are determined, for example, using a Model 510 gel permeation chromatography (Water Associates, Inc., Milford, Mass.) equipped with a high-pressure liquid chromatographic pump, a Waters 486 UV detector and a Waters 2410 differential refractive index detector. Tetrahydrofuran (THF), N,N-dimethylformamide (DMF) or N,N-dimethylacetamide (DMAc) is used as the eluent (1.0 mL/min). Polystyrene or poly(methyl methacrylate) standards having narrow molecular weight distribution were used for calibration.

Methods for making polymers containing a α-amino acid in the general formula are well known in the art. For example, for the embodiment of the polymer of structural formula (I) wherein R⁴ is incorporated into an α-amino acid, for polymer synthesis the α-amino acid with pendant R³ can be converted through esterification into a bis-α,ω-diamine, for example, by condensing the α-amino acid containing pendant R³ with a diol HO—R⁴—OH. As a result, di-ester monomers with reactive α,ω-amino groups are formed. Then, the bis-α,ω-diamine is entered into a polycondensation reaction with a di-acid such as sebacic acid, or bis-activated esters, or bis-acyl chlorides, to obtain the final polymer having both ester and amide bonds (PEA). Alternatively, for example, for polymers of structure (I), instead of the di-acid, an activated di-acid derivative, e.g., bis-para-nitrophenyl diester, can be used as an activated di-acid. Additionally, a bis-di-carbonate, such as bis(p-nitrophenyl) dicarbonate, can be used as the activated species to obtain polymers containing a residue of a di-acid. In the case of PEUR polymers, a final polymer is obtained having both ester and urethane bonds.

More particularly, synthesis of the unsaturated poly(ester-amide)s (UPEAs) useful as biodegradable polymers of the structural formula (I) as disclosed above will be described, wherein

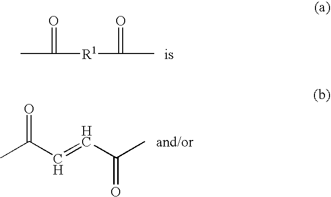

R⁴ is —CH₂—CH═CH—CH₂—. In cases where (a) is present and (b) is not present, R⁴ in (I) is —C₄H₈— or —C₆H₁₂—. In cases where (a) is not present and (b) is present, R¹ in (I) is —C₄H₈— or —C₈H₁₆—.

The UPEAs can be prepared by solution polycondensation of either (1) di-p-toluene sulfonic acid salt of bis(α-amino acid) di-ester of unsaturated diol and di-p-nitrophenyl ester of saturated dicarboxylic acid or (2) di-p-toluene sulfonic acid salt of bis(α-amino acid) diester of saturated diol and di-nitrophenyl ester of unsaturated dicarboxylic acid or (3) di-p-toluene sulfonic acid salt of bis(α-amino acid) diester of unsaturated diol and di-nitrophenyl ester of unsaturated dicarboxylic acid.

The aryl sulfonic acid salts of diamines are known for use in synthesizing polymers containing amino acid residues. The p-toluene sulfonic acid salts are used instead of the free diamines because the aryl sulfonic salts of bis(α-amino acid) diesters are easily purified through recrystallization and render the amino groups as less reactive ammonium tosylates throughout workup. In the polycondensation reaction, the nucleophilic amino group is readily revealed through the addition of an organic base, such as triethylamine, reacts with bis-electrophilic monomer, so the polymer product is obtained in high yield.

Bis-electrophilic monomers, for example, the di-p-nitrophenyl esters of unsaturated dicarboxylic acid, can be synthesized from p-nitrophenyl and unsaturated dicarboxylic acid chloride, e.g., by dissolving triethylamine and p-nitrophenol in acetone and adding unsaturated dicarboxylic acid chloride dropwise with stirring at −78° C. and pouring into water to precipitate product. Suitable acid chlorides included fumaric, maleic, mesaconic, citraconic, glutaconic, itaconic, ethenyl-butane dioic and 2-propenyl-butanedioic acid chlorides. For polymers of structure (V) and (VI), bis-p-nitrophenyl dicarbonates of saturated or unsaturated diols are used as the activated monomer. Dicarbonate monomers of general structure (XIII) are employed for polymers of structural formula (V) and (VI),

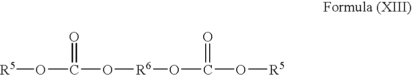

wherein each R⁵ is independently (C₆-C₁₀) aryl optionally substituted with one or more nitro, cyano, halo, trifluoromethyl, or trifluoromethoxy; and R⁶ is independently (C₂-C₂₀) alkylene or (C₂-C₂₀) alkyloxy, or (C₂-C₂₀) alkenylene.

The di-aryl sulfonic acid salts of diesters of α-amino acid and unsaturated diol can be prepared by admixing α-amino acid, e.g., p-aryl sulfonic acid monohydrate and saturated or unsaturated diol in toluene, heating to reflux temperature, until water evolution is minimal, then cooling. The unsaturated diols include, for example, 2-butene-1,3-diol and 1,18-octadec-9-en-diol.

Saturated di-p-nitrophenyl esters of dicarboxylic acid and saturated di-p-toluene sulfonic acid salts of bis-α-amino acid esters can be prepared as described in U.S. Pat. No. 6,503,538 B1.

Synthesis of the unsaturated poly(ester-amide)s (UPEAs) useful as biodegradable polymers of the structural formula (I) as disclosed above will now be described. UPEAs having the structural formula (I) can be made in similar fashion to the compound (VII) of U.S. Pat. No. 6,503,538 B1, except that R⁴ of (III) of U.S. Pat. No. 6,503,538 and/or R¹ of (V) of U.S. Pat. No. 6,503,538 is (C₂-C₂₀) alkenylene as described above. The reaction is carried out, for example, by adding dry triethylamine to a mixture of said (III) and (IV) of U.S. Pat. No. 6,503,538 and said (V) of U.S. Pat. No. 6,503,538 in dry N,N-dimethylacetamide, at room temperature, then increasing the temperature to 80° C. and stirring for 16 hours, then cooling the reaction solution to room temperature, diluting with ethanol, pouring into water, separating polymer, washing separated polymer with water, drying to about 30° C. under reduced pressure and then purifying up to negative test on p-nitrophenol and p-toluene sulfonate. A preferred reactant (IV) of U.S. Pat. No. 6,503,538 is p-toluene sulfonic acid salt of Lysine benzyl ester, the benzyl ester protecting group is preferably removed from (II) to confer biodegradability, but it should not be removed by hydrogenolysis as in Example 22 of U.S. Pat. No. 6,503,538 because hydrogenolysis would saturate the desired double bonds; rather the benzyl ester group should be converted to an acid group by a method that would preserve unsaturation. Alternatively, the lysine reactant (IV) of U.S. Pat. No. 6,503,538 can be protected by a protecting group different from benzyl that can be readily removed in the finished product while preserving unsaturation, e.g., the lysine reactant can be protected with t-butyl (i.e., the reactant can be t-butyl ester of lysine) and the t-butyl can be converted to H while preserving unsaturation by treatment of the product (II) with acid.

A working example of the compound having structural formula (I) is provided by substituting p-toluene sulfonic acid salt of bis(L-phenylalanine) 2-butene-1,4-diester for (III) in Example 1 of U.S. Pat. No. 6,503,538 or by substituting di-p-nitrophenyl fumarate for (V) in Example 1 of U.S. Pat. No. 6,503,538 or by substituting the p-toluene sulfonic acid salt of bis(L-phenylalanine) 2-butene-1,4-diester for III in Example 1 of U.S. Pat. No. 6,503,538 and also substituting bis-p-nitrophenyl fumarate for (V) in Example 1 of U.S. Pat. No. 6,503,538.

In unsaturated compounds having either structural formula (I) or (IV), the following hold. An amino substituted aminoxyl (N-oxide) radical bearing group, e.g., 4-amino TEMPO, can be attached using carbonyldiimidazol, or suitable carbodiimide, as a condensing agent. Bioactive agents, as described herein, can be attached via the double bond functionality.

The biodegradable PEA, PEUR and PEU polymers can contain from one to multiple different bioactive agents and α-amino acids per polymer molecule and preferably have weight average molecular weights ranging from about 20,000 Da to about 300,000 Da, for example 65,000 Da to about 130,000 Da; these polymers and copolymers typically have intrinsic viscosities at 25° C., as determined by standard viscosimetric methods, ranging from 0.3 to 4.0, for example, ranging from 0.5 to 3.5.

PEA and PEUR polymers contemplated for use in the practice of the invention can be synthesized by a variety of methods well known in the art. For example, tributyltin (IV) catalysts are commonly used to form polyesters such as poly(ε-caprolactone), poly(glycolide), poly(lactide), and the like. However, it is understood that a wide variety of catalysts can be used to form polymers suitable for use in the practice of the invention.

Such poly(caprolactones) contemplated for use have an exemplary structural formula (XIV) as follows:

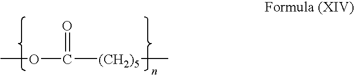

Poly(glycolides) contemplated for use have an exemplary structural formula (XV) as follows:

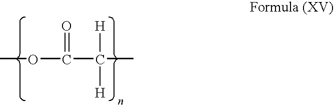

Poly(lactides) contemplated for use have an exemplary structural formula (XVI) as follows:

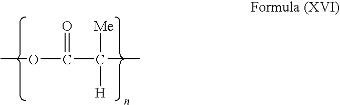

An exemplary synthesis of a suitable poly(lactide-co-ε-caprolactone) including an aminoxyl moiety is set forth as follows. The first step involves the copolymerization of lactide and ε-caprolactone in the presence of benzyl alcohol using stannous octoate as the catalyst to form a polymer of structural formula (XVII).

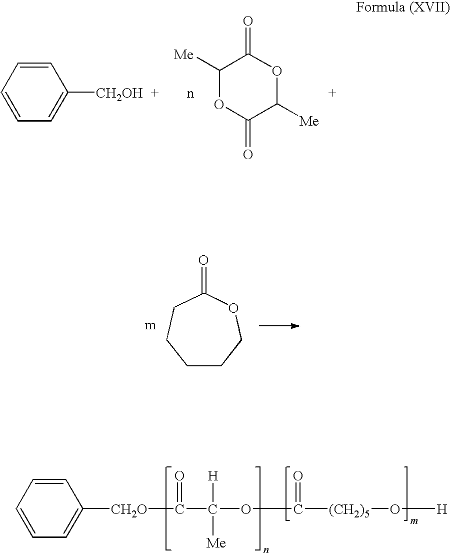

The hydroxy terminated polymer chains can then be capped with maleic anhydride to form polymer chains having structural formula (XVIII):

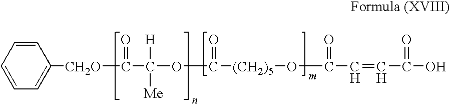

At this point, 4-amino-2,2,6,6-tetramethylpiperidine-1-oxy can be reacted with the carboxylic end group to covalently attach the aminoxyl moiety to the copolymer via the amide bond which results from the reaction between the 4-amino group and the carboxylic acid end group. Alternatively, the maleic acid capped copolymer can be grafted with polyacrylic acid to provide additional carboxylic acid moieties for subsequent attachment of further aminoxyl groups.

In unsaturated compounds having structural formula (VII) for PEU, the following hold: An amino substituted aminoxyl (N-oxide) radical bearing group e.g., 4-amino TEMPO, can be attached using carbonyldiimidazole, or suitable carbodiimide, as a condensing agent. Additional bioactive agents, and the like, as described herein, optionally can be attached via the double bond functionality.

For example, the invention high molecular weight semi-crystalline PEUs having structural formula (VII) can be prepared inter-facially by using phosgene as a bis-electrophilic monomer in a chloroform/water system, as shown in the reaction scheme (2) below:

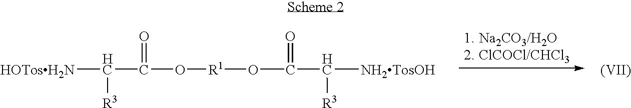

Synthesis of copoly(ester ureas) (PEUs) containing esters of adirectional amino acids, such as L-Lysine esters, and having structural formula (VII) can be carried out by a similar scheme (3):

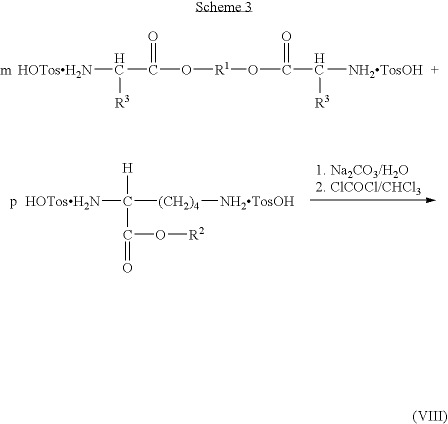

A 20% solution of phosgene (ClCOCl) (highly toxic) in toluene, for example (commercially available (Fluka Chemie, GMBH, Buchs, Switzerland), can be substituted either by diphosgene (trichloromethylchloroformate) or triphosgene (bis(trichloromethyl)carbonate). Less toxic carbonyldiimidazole can be also used as a bis-electrophilic monomer instead of phosgene, di-phosgene, or tri-phosgene.

### General Procedure for Synthesis of PEUs

It is necessary to use cooled solutions of monomers to obtain PEUs of high molecular weight. For example, to a suspension of di-p-toluenesulfonic acid salt of bis(α-amino acid)-α,ω-alkylene diester in 150 mL of water, anhydrous sodium carbonate is added, stirred at room temperature for about 30 minutes and cooled to about 2-0° C., forming a first solution. In parallel, a second solution of phosgene in chloroform is cooled to about 15-10° C. The first solution is placed into a reactor for interfacial polycondensation and the second solution is quickly added at once and stirred briskly for about 15 min. Then chloroform layer can be separated, dried over anhydrous Na₂SO₄, and filtered. The obtained solution can be stored for further use.

All the exemplary PEU polymers fabricated were obtained as solutions in chloroform and these solutions are stable during storage. However, some polymers, for example, 1-Phe-4, become insoluble in chloroform after separation. To overcome this problem, polymers can be separated from chloroform solution by casting onto a smooth hydrophobic surface and allowing chloroform to evaporate to dryness. No further purification of obtained PEUs is needed.

As described above, the invention compositions contain a stabilizing polymer with lipophilic properties, for example at least one or a blend of the PEA, PEUR and PEU stabilizing polymers of formulas (I and IV-VIII. As is described in greater detail below, such lipophilic polymer chains are incorporated into the same liquid solution as the lipids (e.g., of Classes I, II, III, and IV described herein) by dissolving between about 1%-90% by weight of the stabilizing polymer in such lipid-containing solution prior to emulsification of such first solution into a second liquid to obtain the liposomal particles.

### The Liposomal Particles

The liposomal particles in the invention composition comprise natural or synthetic lipids and other compounds that act like lipids (i.e., “lipid-acting compounds”), which aid in stabilization of the bioactive agent to be delivered by the composition. The terms “lipids” and “lipid-acting compounds” as used herein to describe components used in the invention compositions means compounds that (a) can form spontaneously into bilayer vesicles in water, as exemplified by the phospholipids, or (b) can be stably incorporated into lipid bilayers, with a hydrophobic moiety thereof in contact with the interior, hydrophobic region of a bilayer membrane, and the head group moiety oriented toward the exterior, polar surface of the membrane. However, the liposomal particles in the invention compositions do not maintain the particle structure by means of separate aqueous-filled compartments of the type found in typical prior art liposomes, and thus can be lyophilized without destruction of the polymer-stabilized lipid layers.

Examples of phospholipids suitable for use in the making of the invention composition include phosphatidylcholine, phosphatidylethanolamine, phosphatidic acid, phosphatidylinositol, and sphingomyelin comprising a polar or non-polar head group, two hydrocarbon chains typically between about 14-22 carbon atoms in length, and have varying degrees of unsaturation. Additional diacyl-chain lipids for use making the invention compositions include diacyl glycerol, phosphatidyl ethanolamine (PE), phosphatidylglycerol (PG), and the like. These lipids and phospholipids whose acyl chains have varying degrees of saturation can be obtained commercially or prepared according to published methods.

The vesicle-forming lipid(s) can be selected to achieve a specified degree of fluidity or rigidity, to control the stability of the liposomal particle in serum and to control the rate of release of the sequestered bioactive agent in the liposomal formulation. The rigidity of the liposomal particle, as determined by the vesicle-forming lipid, may also play a role in fusion of the liposomal particle to a target cell. Liposomal particles having a more rigid lipid bilayer, or a liquid crystalline bilayer, are achieved by incorporation of a relatively rigid lipid, e.g., a lipid having a relatively high phase transition temperature, e.g., up to 60° C. Rigid, e.g., saturated, lipids contribute to greater membrane rigidity in the lipid bilayer. Other lipid components, such as cholesterol, are also known to contribute to membrane rigidity in lipid bilayer structures. On the other hand, lipid fluidity can be achieved by incorporation of a relatively fluid lipid, typically one having a lipid phase with a relatively low liquid to liquid-crystalline phase transition temperature, for example, at or below room temperature.

In certain embodiments, the lipids forming the lipid bilayer(s) in the vesicle, i.e., liposomal particle, can also include cationic lipids, which have a lipophilic moiety, such as a sterol or an acyl or diacyl chain, and where the lipid has an overall net positive charge. Preferably, in this case, the head group of the lipid carries the positive charge. Exemplary cationic lipids include 1,2-dioleyloxy-3-(trimethylamino)propane (DOTAP); N-[1-(2,3,-ditetradecyloxy)propyl]-N,N-dimethyl-N-hydroxyethylammonium bromide (DMRIE); N-[1-(2,3,-dioleyloxy)propyl]-N,N-dimethyl-N-hydroxy ethylammonium bromide (DORIE); N-[1-(2,3-dioleyloxy)propyl]-N,N,N-trimethylammonium chloride (DOTMA); 3.beta.[N—(N′,N′-dimethylaminoethane)carbamoly]cholesterol (DC-Chol); and dimethyldioctadecylammonium (DDAB).

The vesicle-forming lipid may also be a neutral lipid, such as dioleoylphosphatidyl ethanolamine (DOPE) or an amphipathic lipid, such as a phospholipid, derivatized with a cationic lipid, such as polylysine or other polyamine lipids. For example, the neutral lipid (DOPE) can be derivatized with polylysine to form a cationic lipid.

Presently preferred synthetic and naturally-occurring vesicle-forming lipids, and lipid-acting molecules, include the following classes (It should be noted that there is considerable overlap between the classes, particularly Class I and Class II vesicle-forming lipids):

Class I: Non-swelling, non-polar stabilizers
Non-ionized fatty acids, such as oleic acid and oleic acid methyl esters; triglycerides, such as cottonseed oil (triolein); and cholesterol esters, such as palmitoyl cholesterol ester, and the like.
   Class II: Polar but insoluble swelling amphiphiles
Amphiphilic lipids, including phospholipids, glycolipids, proteolipids, and the like; amphipathic biologics (including membrane-associated proteins); and amphipathic peptides (including phosphopeptides, glycopeptides, short (e.g. C₅-C₁₀) hydrocarbon chain lipopeptides); lecithin-phosphatidylcholine, such as phosphatidylethanolamine, and phosphatidylserine; mono-glycerides; ionized and/or partially ionized fatty acids, and the like.
   Class III: soluble amphiphiles (with liotropic mesomorphism)
Short hydrocarbon chain (e.g. C₅-C₁₀) lipids, such as phospholipids, glycolipids, proteolipids, and the like; amphipathic biologics (including membrane-associated proteins); and amphipathic peptides (including phosphopeptides, glycopeptides, short hydrocarbon chain lipopeptides); amphiphilic polymers (such as, poly-ols, polyethers, polyglycols, polysaccarides): for example, Poly (vinyl alcohol), poly(ethylene) glycol, hydroxypropyl methyl cellulose, mannitol, and the like; starch and chitosan based polymers, alginates, pectins, hyaluronates, and heparin, all are hydrophilic but can be used in polymeric amphiphiles; Polyethers, such as PEG; and polymers for enteric coating of liposomal compositions (e.g. Sureteric™, pHthalavin™, Acryl-eze MP™, Eurdragit L100®, Opadry Enteric™ or Cellacefate™).
   Class IV: soluble amphiphiles as liposomal particle stabilizers
Cholesterol, and cholesterol based compounds, such as bile salts e.g., sodium cholate, sodium taurocholate, and the like.

Lipid vesicles containing entrapped bioactive agents can be prepared using well-known methods, such as double emulsion and evaporation, hydration of a lipid film, reverse-phase evaporation, and solvent infusion. The bioactive agent to be delivered can be either included in the lipid phase, in the case of a lipophilic bioactive agent, or in the hydration medium, in the case of a water-soluble bioactive agent. Previously described techniques for producing liposomal particles include the following: U.S. Pat. Nos. 4,522,803—Lenk, 4,310,506—Baldeschwieler, 4,235,871—Papahadjopoulos, 4,224,179—4,078,052—Papahadjopoulos, 4,394,372—Taylor, 4,308,166—Marchetti, 4,485,054—Mezei, and 4,508,703—Redziniak. For a review of various methods of liposome preparation, refer to Szoka, et al. (Ann. Rev. Biophys. Bioeng., 9:467-508, 1980).

However, the liposomal particles made by the invention methods are not true liposomes because the structure of the particles is organized by the stabilizing polymer with lipophilic properties, which permeates the product liposomal particles throughout, rather than by the presence of separate lipid and aqueous compartments. In fact, the invention polymer-stabilized liposomal particles do not possess separate lipid and aqueous compartments (although whole water molecules are preferably hydrogen bonded to the polymer even when the particles have been lyophilized). Therefore the invention composition can be lyophilized without loss of the particle structure and without loss of native activity of the sequestered bioactive agent, such as a biologic. As described in detail below, the bioactive agent, whether hydrophobic or hydrophilic, is sequestered in concert by the lipids and the stabilizing polymer in the invention compositions. In embodiments of the invention wherein the stabilizing polymer provides free functional groups, prior to combining the components for formation of particles, a bioactive agent, e.g., a macromolecular biologic, can be preconjugated to the stabilizing polymer. In general, the stabilizing polymer not only stabilizes the structure of the particles during and after their formation, but also stabilizes the folded structure of a biologic to retain native activity of such complex molecules within the invention compositions and for release therefrom.

For example, the amide groups at the ends of the polymer chain or a polymer bearing carboxyl groups, such as those of structural formulas (IV, VI and VIII), can readily react with numerous complementary functional groups that can be used to covalently attach a bioactive agent (e.g., an affinity moiety, a peptidic antigen, an insulin molecule, or other macromolecular biologic) to the biodegradable polymer. In another example, an amino moiety in a peptide can readily react with a carboxyl group in these polymers to covalently bond a peptide to the polymer via the resulting amide group. Since the homopolymers of structural formulas (I, V, and VII) contain free functional groups only at the two ends of the polymer chain, whereas the co-polymers of structural formulas (IV, VI and VIII) have a free carboxyl moiety in each adirectional amino acid-based moiety, the co-polymers are suitable for attaching a larger load of bioactive agent than are the homo-polymers.

### Polymer—Bioactive Agent or Macromolecular Biologic Conjugation

Accordingly, in one embodiment, the polymers used to make the invention polymer-stabilized liposomal compositions as described herein have one or more macromolecular biologic or bioactive agent directly linked to the polymer. The residues of the polymer can be linked to the residues of the one or more macromolecular biologics or bioactive agents. For example, one residue of the polymer can be directly linked to one residue of the macromolecular biologic or bioactive agent. The polymer and the macromolecular biologic or bioactive agent can each have one open valence. Alternatively, more than one, multiple, or a mixture of macromolecular biologics and bioactive agents having different therapeutic or palliative activity can be directly linked to the polymer. However, since the residue of each macromolecular biologic or bioactive agent can be linked to a corresponding residue of the polymer, the number of residues of the one or more macromolecular biologic or bioactive agents can correspond to the number of open valences on the residue of the polymer.

As used herein, a “residue of a polymer” refers to a radical of a polymer having one or more open valences. Any synthetically feasible atom, atoms, or functional group of the polymer (e.g., on the polymer backbone or pendant group) of the present invention can be removed to provide the open valence, provided bioactivity is substantially retained when the radical is conjugated to a residue of a bioactive agent. Additionally, any synthetically feasible functional group (e.g., carboxyl) can be created on the polymer (e.g., on the polymer backbone or pendant group) to provide the open valence, provided bioactivity is substantially retained when the radical is conjugated to a residue of a bioactive agent. Based on the linkage that is desired, those skilled in the art can select suitably functionalized starting materials that can be derived from the polymer of the present invention using procedures that are known in the art.

As used herein, a “residue of a compound of structural formula (*)” refers to a radical of a compound of polymer formulas (I) and (IV-VIII) as described herein having one or more open valences. Any synthetically feasible atom, atoms, or functional group of the compound (e.g., on the polymer backbone or pendant group) can be removed to provide the open valence, provided bioactivity is substantially retained when the radical is conjugated to a residue of a bioactive agent. Additionally, any synthetically feasible functional group (e.g., carboxyl) can be created on the compound of formulas (I) and (IV-VIII) (e.g., on the polymer backbone or pendant group) to provide the open valance, provided bioactivity is substantially retained when the radical is conjugated to a residue of a bioactive agent. Based on the linkage that is desired, those skilled in the art can select suitably functionalized starting materials that can be derived from the compound of formulas (I) and IV-VIII) using procedures that are known in the art.

For example, the residue of a bioactive agent can be linked to the residue of a compound of structural formula (I) or (IV) through an amide (e.g., —N(R)C(═O)— or —C(═O)N(R)—), ester (e.g., —OC(═O)— or —C(═O)O—), ether (e.g., —O—), amino (e.g., —N(R)—), ketone (e.g., —C(═O)—), thioether (e.g., —S—), sulfinyl (e.g., —S(O)—), sulfonyl (e.g., —S(O)₂—), disulfide (e.g., —S—S—), or a direct (e.g., C—C bond) linkage, wherein each R is independently H or (C₁-C₆) alkyl. Such a linkage can be formed from suitably functionalized stabilizing polymers and bioactive agents, as described herein, using synthetic procedures that are known in the art. Based on the linkage that is desired, those skilled in the art can select suitably functional starting material that can be derived from a residue of a compound of structural formula (I) or (IV) and from a given residue of a bioactive agent using procedures that are known in the art. The residue of the bioactive agent or adjuvant can be linked to any synthetically feasible position on the residue of a compound of structural formula (I) or (IV). Additionally, the invention also provides compounds having more than one residue of a bioactive agent directly linked to a compound of structural formula (I) or (IV).

The number of molecules of bioactive agents that can be linked to the polymer molecule can typically depend upon the molecular weight of the polymer and the equivalents of functional groups incorporated. For example, for a compound of structural formula (I), wherein n is about 5 to about 150, preferably about 5 to about 70, up to about 150 macromolecular biologics or bioactive agent molecules (i.e., residues thereof) can be directly linked to the polymer (i.e., residue thereof) by reacting the bioactive agent with free functional groups of the stabilizing polymer. In unsaturated polymers, the bioactive agents can also be reacted with double (or triple) bonds in the polymer.

In embodiments of the invention in which a bioactive agent is conjugated to the stabilizing polymer, such attachment is performed as described herein, prior to incorporation of the stabilizing polymer into the liposomal particles. Therefore it is important that the conditions for synthesis of the liposomal particles be selected such that the attached bioactive agent, e.g., a biologic, will not be denatured during formation of the liposomal particles.

In one embodiment in which the bioactive agent is a biologic, such as a macromolecular biologic, whose biologic activity depends upon maintenance of a particular three dimensional structure, the preferred stabilizing polymer is a PEA, PEUR or PEUR described by structural formulas (I) and (IV-VIII). These polymers are particularly suited for this purpose because they contain alternating lipophilic and polar segments, as does, in general, the surface of folded biologics, such as proteins. Moreover the polymer polar segments, being whole amino acid residues, are capable of hydrogen bonding both to the biologic and to biologic-bound water molecules, a factor that is known in the art to be essential for the preservation of the folded structure, and hence, activity, of a biologic.

### Programmed Bio-Transformation

In compositions intended for administration in vivo via a particular route, e.g., the gastrointestinal tract, or nasally, it is preferred that that the lipids and lipid-acting compounds included in the invention compositions are both naturally occurring and are selected to mimic the role of native molecules whose function is (i) wholly or partially to stabilize native molecules within the particular route of administration within the body and/or (ii) wholly or partially to mediate the trafficking of native molecules within the body.

For example, for oral delivery of a biologic, such as insulin, a fatty acid, a triglyceride and a bile salt can be selected to enhance stability of the liposomal particles. These classes of molecule are used by the body in the generation and stabilization of emulsions of fatty food material during digestion.

Accordingly, in one embodiment, the invention provides polymer-stabilized liposomal compositions that are intended for delivery of a biologic to the gastrointestinal tract, for example orally. In this embodiment, the polymer-stabilized liposomal composition comprises lipid or lipid-acting compounds selected from the Class I, II, III and IV compounds, preferably at least one such compound from each of Classes I, II, III and IV, including at least one bile salt, an endogenous permeation enhancer. Since bile salts contain a substantial lipid component, the bile salt will partition into the lipid bilayer(s) of the liposomal particle. In this embodiment, polymer-stabilized liposomal compositions, as described herein, can be used to orally deliver the bioactive agent, in concentrated amounts to the microvilli of the intestine for absorption by protecting it from enzymatic degradation, such as proteolysis. Under normal physiological conditions in the intestine, absorption of a protein or protein fragment by the columnar epithelium is very low. In this alternative embodiment of the invention, inclusion of one or more bile salts in the liposomal particles that sequesters the biologic, enhance permeability across the intestinal wall, perhaps due to the presence of sterol-like molecules at the surface of the liposomal particles. Thus, the bile salt in the invention liposomal particle compositions contributes stability to and protects the biologic within the liposomal particle as it travels through the lumen of the intestine. In addition, the bile salt also enhances rapid release of the biologic from the liposomal particles when subjected to the physiological conditions of the brush border of the intestine.

In fact, it is expected that the released biologic will be protected by spontaneous formation of micelles around the biologic and that absorption of the biologic will be enhanced by formation of chylomicron-like particles for delivery of the biologic through the mucosal cells of the villi. Whatever the exact mechanism, a concentrated bolus of a biologic can be quickly released by the liposomal particles into the mucous and glycocalyx layers coating the simple columnar epithelium. From there, the bile salt-coated biologic efficiently diffuses through the epithelial cells and lamina propria as chylomicron-like particles and is rapidly transported by blood flow through the hepatic portal vein to the hepatocytes of the liver when the liposomal particle composition is administered to the gastrointestinal tract, for example, orally.

In the embodiment of the invention in which one or more bile salts are in the lipids of the liposomal particles that sequester the biologic, advantage is taken of a major circulatory pathway, the enterohepatic circulatory pathway, for uptake of the biologic from the small and large intestine to the liver. This pathway is important in recycling bile salts through the gut to aid in the digestion and absorption of food. The transport of intact biologically active macromolecules from the intestinal lumen into the blood circulation is a unique phenomenon which differs from the regular process of food digestion and absorption. Intestinal absorption of bioactive peptides and various proteins has been reported (Ziv, E., et al. Biochemical Pharmacology (1987) 36(7):1035-1039). It has been shown that protection against proteolysis is the first step involved in keeping polypeptides and proteins intact in the “hostile” intestinal lumen (See references in Ziv, supra). The second step entails alteration of the mechanisms responsible for selective absorption of small molecules to enable absorption of high molecular weight molecules. Since they are endogenous, these natural and specialized amphipathic permeation enhancers are less likely to produce severe side effects in the individual than are other types of amphipathic molecules.

Bile is a hepatic secretion that appears to have two principal functions: first, to promote the digestion and absorption of lipid from the intestine, and second, to enhance elimination of many endogenous and exogenous substances from the blood and liver that are not excreted through the kidneys (Strange, R. C., Hepatic bile flow, Physiological Reviews, (1984) 64(4):1055-1102). Bile salts, a major constituent of bile, have a concentration in bile between 2 and 45 mM and are acidic sterols, which in mammals are based on the C₂₄ compound, cholic acid. The bile salts useful in the invention include the commonly occurring bile salts based on cholic acid: cholate, chenodeoxycholate and lithocholate, which differ in the number of hydroxyl groups on the cholic acid ring structure. The natural bile salts optionally used in the invention liposomal particle compositions will be reused by the liver for its own production of bile. Re-absorption of such salts occurs mainly in the duodenum and terminal ileum and, after passage across the cells of the small intestinal wall, bile salts return to the liver via the portal circulation. In humans 99% of the bile salt pool is maintained within the enterohepatic circulation and during each 24-h period approximately 40 g (100 mmol) of bile salt is removed from the portal blood by the liver. Excess bile salts are eliminated through the bowel. (Strange, supra).

Since bile salts reach the liver predominantly via the portal vein, it can be expected that addition of at least one bile salt to the invention liposomal particle composition will significantly contribute to the delivery of the biologic contained therein to hepatocytes, which are arranged in sheets one cell thick and are situated between the afferent and efferent blood supplies. The composition will first contact the sinusoidal surface of the liver cells, which is the site of receptor systems for several hormones, including insulin, glucagon, and bile salts. Microvilli on the sinusoidal surface considerably increase the surface area available for an exchange of molecules between blood and liver cells.

Furthermore, it is known that micro-emulsions of dietary fats can pass between the micro-invaginations (villi) of the lining of the small intestine and perhaps be trapped between them. However, to pass into the intestinal wall these micro-emulsions must be further emulsified into nano-emulsions that can become trapped between microvilli that cover the surface of the villi. Transition of such micro-emulsions to nano-emulsions is mediated by body enzymes during normal digestion. For example, gastric, pancreatic and intestinal lipases degrade micro-emulsified dietary lipids to fatty acids. A portion of these de novo fatty acids is lost from the micro-emulsion droplets, reducing the bulk eventually to a nano-emulsion. The remainder of the de novo fatty acid molecules remain in the shrinking droplets and contribute stability thereto because (i) in the stomach acid, the fatty acid molecules are neutralized by protonation, thus lowering their aqueous solubility, (ii) the fatty acid molecules act as digestive enzyme inhibitors, and (iii) the fatty acids become bound to the nano-emulsion by esterification to cholesterol.

Accordingly, for oral delivery of a biologic, the lecithin group of natural phospholipids (phosphatidylcholines) (Class II lipids and lipid-acting molecules) can be selected as a particle-bound surfactant to promote emulsification during fabrication of the composition. This particular set of Class II phospholipids also function in vivo as natural substrates for pancreatic and intestinal phospholipases, which cleave phospholipids to produce de novo fatty acids. Thus, it is believed that invention polymer stabilized liposomal compositions comprising such Class II lipids and lipid-acting molecules are naturally transformed within the stomach and then the small intestine of mammals from micro-particles to nano-particles suitable for entrapment by micro-villi, thereby delivering polymer-conjugated biologic in the invention composition (e.g. insulin) into the intestinal wall.

Other lipids and lipid-acting molecules used in preparation of the invention liposomal particles for oral delivery of biologics, the fatty acid, triglycerides (Class I) and bile salts (Class IV) can be selected, not only to contribute the desirable properties for preparation of the composition as described herein, but also to hinder and/or inhibit the action of digestive enzymes, such as lipases and other hydrolases. Thus, these Class I and Class IV lipids and lipid-acing molecules protect the polymer stabilized liposomal nano-formulation during digestion. In addition, these lipids and lipid-acting molecules facilitate and/or mediate transport of the biologic through the intestinal wall for release into the portal blood system.

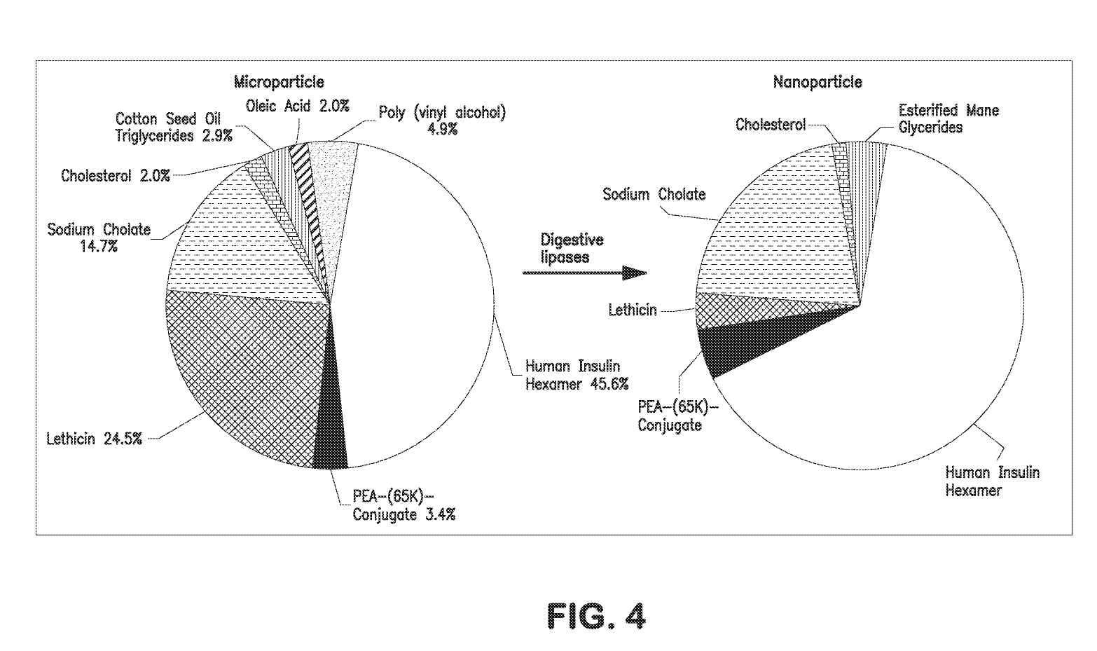

A schematic representation of the biological transformation of an invention composition formulated for oral delivery of the biologic insulin and prepared as in Example 1 herein is illustrated schematically in FIG. 4.

### The Preferred Process for Making the Invention Polymer-Stabilized Liposomal Compositions for Stabilization of a Biologic, e.g., a Macromolecular Biologic

Step 1—Concentration and stabilization of a biologic conjugated to the stabilizing polymer First a lipophilic polymer is used to stabilize a monomer of a biologic attached thereto as described above. This stabilization is achieved by first solubilizing both the biologic and the polymer in a solvent, or mixture of miscible solvents, that is sufficiently hydrophobic to solubilize the polymer but is sufficiently polar to solubilize the biologic in its bio-active, folded form.

Although any of the disclosed polymers can be used for this purpose, the preferred polymers useful for stabilizing both the activity of a biologic and the lipids layers in the liposomal particles are those described by structural formulas (I, V and VII) or by structural formulas (IV, VI and VIII) in which at least some of the R³s are H. Either one or both of the R³ s and the carboxylates of the adirectional amino acid residues incorporated into the co-polymer are useful to attach a molecule of a synthetic or natural biologic. For example, a monomer of a biologic can be conjugated to the carboxylate of adirectional lysine in such co-polymers.

The attached biologic is then concentrated in the presence of polymer under conditions that promote self stabilization of the biologic via oligomerization and/or crystallization. For example, free insulin can be concentrated from solution in the presence of a co-PEA polymer-insulin monomer conjugate under conditions conducive to formation of insulin hexamers and crystallization of insulin hexamers. Concentration and stabilization of the biologic is achieved by the controlled replacement of the solvent or mix of miscible solvents with a second liquid phase that promotes oligomerization and/or crystallization of the biologic. For example, the first solvent or mix of miscible solvents can be an organic phase and the second phase can be aqueous. This two phase system will generally be used for packaging and delivery of biologics via the invention polymer-stabilized liposomal formulations. The second liquid phase can optionally contain additive molecules that are known or demonstrated to preserve and/or promote oligomerization and/or crystallization of the biologic as described herein, such as atoms of transition metals or calcium.

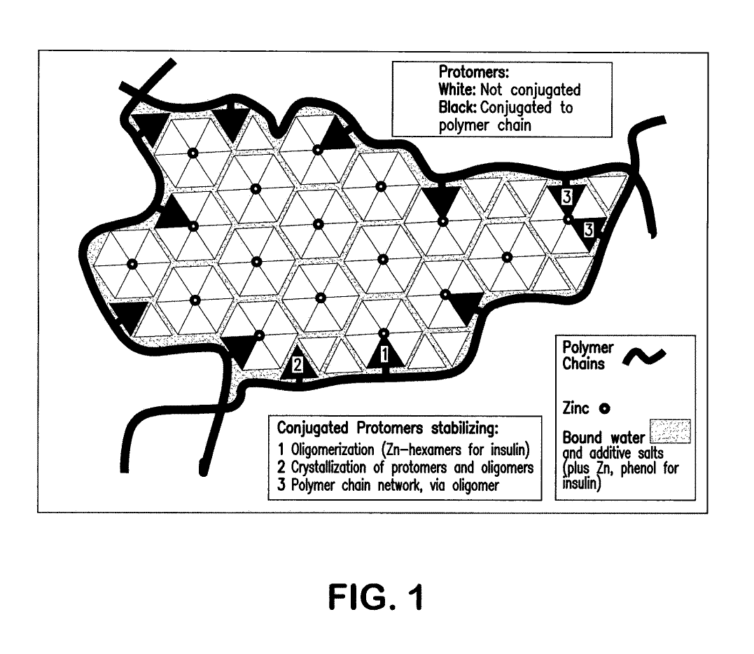

The resulting concentrate is lyophilized, during which the initial solvent or mix of miscible solvents is evaporated, leaving an aqueous, freeze-dried powder with chemical structure as illustrated in FIG. 1 wherein the biologic conjugated to the polymer is insulin monomers. The concentration of the biologic in the product composition is in the range from about 5% to about 60% by weight.

Step 2—Preparation of the liquids for emulsification: First a concentrated solution of the freeze-dried powder prepared above and the lipids and lipid-acting compounds for the lipid phase of the liposomal particles are dissolved in a solvent A. These lipids and lipid-acting compounds possess at least one of the following properties:
1) Class I: organic based stabilizers (non-swelling, non-polar) in concert with the polymer, or the polymer-insulin monomer conjugate, stabilize the cargo biologic within solvent A;
2) Class II: organic based surfactants (swelling amphiphiles) stabilize the micro- and/or nano-emulsified droplets of solvent A within liquid B and so aid emulsification by acting as surfactant at the interface between solvent droplets A and liquid B.

One or more compounds from Class III (aqueous based surfactants and/or coatings) can be added to liquid B prior to emulsification. These excipients are required to possess the ability to stabilize the micro- and/or nano-emulsified droplets of solvent A within liquid B to not only aid emulsification by acting as surfactant at the interface between liquid B and solvent droplets A, but also to stabilize the liposomal particles in the presence of residual liquid B during Step 3.

For emulsification, solvent A and liquid B do not have to be immiscible. For example, solvent A can be an organic phase, and liquid B can be aqueous. However, Solvent A for Step 2 is required to possess the same properties (e.g., lipid or aqueous) as the first solvent in step 1, although the two need not be identical. Thus, solvent A in the emulsification must be a liquid in which all of the contents of the liposomal formulation except the aqueous based surfactants and/or coatings of Class III have significant solubility, for example an organic solvent such as HFIP and/or DMSO. Solvent B must be a liquid, for example aqueous, in which the aqueous based surfactants and/or coatings of Class III have significant solubility, but all of the other contents of the formulation do not.

Emulsification down to small, micro- and/or nano-particles is achieved by energy input in the form of a shear force, such as is provided by agitation by stirring, sonication, and the like. Stabilization of micro- and/or nano-droplets of solvent A by the presence of the stabilizing polymer or by the co-polymer-biologic monomer conjugate is an important feature of this embodiment of the invention. After emulsification, A diffuses out of the emulsified droplets into B, because here A and B are miscible. Without the presence of the stabilizing polymer, the Classes I, II and IV lipids and lipid-acting molecules within the droplets A would form meta-stable micellar liposomal particles as A diffuses into B. However, in the presence of the stabilizing polymer, these lipids remain bound together as polymer stabilized liposomal particles, as A diffuses into B.

Moreover, it might be assumed that because most folded biologics are water soluble, the folded biologic would be lost from the emulsified droplets as A diffuses into B (because here A and B are miscible). However, it has been shown (see Example 1, herein) that insulin molecules and the lipids remain together because they are all stabilized together by the polymer.

Step 3-Evaporation and lyophilization—The emulsion of micro- and/or nano-particles is concentrated by evaporation under reduced pressure so that solvent A is lost by evaporation, leaving an aqueous freeze-dried powder of polymer-stabilized liposomal particles.

One or more Class IV lipid or lipid-acting molecules, as defined herein, can be added to solvent A prior to emulsification to stabilize the biologic in the presence of residual liquid B during the evaporation.

One or more Class III lipid-acting molecules, as defined herein, can be added to liquid B prior to emulsification, not only to aid in emulsification by acting as surfactant at the interface between liquid B and solvent droplets A, but also to stabilize the liposomal particles in the presence of residual liquid B during evaporation and lyophilization.

Step 4-Reconstitution of the lyophilized powder—For use, the lyophilized powder from Step 3 is reconstituted by dissolving into a type B liquid. The presence of Class III molecules in the formulation during reconstitution will promote aqueous dispersal of the polymer-stabilized liposomal particles. In vivo, such type B liquids are compatible with and are also provided by the biological milieu.

### The Bioactive Agents

The invention polymer-stabilized liposomal composition is for use in delivering a bioactive agent to a subject and comprises at least one bioactive agent. The bioactive agent may be any of a large number of therapeutic or palliative agents that can be entrapped in the liposomal particles, including water-soluble agents that can be stably entrapped by a polar segment of the stabilizing polymer in the particles, lipophilic agents that stably partition into the lipid phase, and agents, such as targeting biologics, that can be stably attached, e.g., by electrostatic or covalent attachment to functional groups on the stabilizing polymer molecules, as will be described below in greater detail. In this case, the bioactive agent, typically a biologic (e.g., a targeting molecule) will be displayed on the outer surface of the liposomal particle and the stabilizing polymer not only stabilizes the liposomal particle, but stabilizes the bioactive agent as well.

As used herein, the term “biologic agent” includes various molecules that affect a biological process. The sequestered bioactive agent may also be a reporter molecule or diagnostic molecule, such as an enzyme or a fluorophore, for use in diagnostic assays. Examples of water-soluble bioactive agents include small, water-soluble organic compounds, such as drugs, adjuvants, and various excipients known in the art. The term “biologic”, as used herein, is a subset of “biologic agents” and refers to molecules that are naturally produced or synthetic and which participate in a biological process, such as peptidic antigens, peptides, proteins, protein fragments, DNA plasmids, oligonucleotides and gene fragments. The terms “peptide” or “peptidic antigen” as used herein distinguish peptide sequences and derivatives thereof having a molecular weight of less than 10,000 daltons from larger amino acid-containing molecules, which may be composed of one or more peptide chains, and which are referred to as “proteins”.

A “macromolecular biologic” as the term is used herein refers to a subset of “biologics” wherein the macromolecules are composed of one or more polymeric chains, forming a three-dimensional structure held together by non-covalent forces, both hydrophobic and ionic; such as is observed in native or synthetically produced proteins and polynucleic acids. Examples of macromolecular biologics include oligomers, such as dimers and sextets of a protein or polynucleic acid, and crystals thereof. In such macromolecular configurations, the biologic molecules preserve the three-dimensional structure necessary for biological activity or the ability to resume such three-dimensional structure upon dissolution of the invention liposomal particle composition.

The liposomal particles having an entrapped biologic agent may deliver the entrapped biologic agent systemically, for example by degradation of the liposomal particle in the blood stream, by fusion to target cells, or by binding of a targeting affinity ligand contained in the liposomal particle to a receptor of a target cell.

### Liposomal Particles

In one embodiment of the invention, the liposomal particles are for use in administering a bioactive agent in vivo to a subject at a controlled rate and include a bioactive agent entrapped by the liposomal particle. The sequestered bioactive agent may be any of a large number of bioactive agents as described herein and as known in the art, that can be entrapped in lipid vesicles, including water-soluble bioactive agents, lipophilic bioactive agents, and agents that can be stably attached, e.g., by covalent attachment to functional groups in the stabilizing polymer included in the lipid bilayer(s) and thus are present on the outer surfaces of the liposomal particles. Exemplary water-soluble compounds include small, organic compounds, peptides, proteins, DNA plasmids, oligonucleotides and gene fragments. By judicious selection of the building blocks of a PEA, PEUR or PEU polymer the electrical properties of the stabilizing polymer can be tailored to be conducive to entrapment of a particular bioactive agent. DNA, for example, is negatively charged and is readily entrapped in a lipid bilayer providing a cationic component.

While a bioactive agent can be entrapped within the liposomal particles without chemical linkage to the stabilizing polymer, it is also contemplated that, in certain embodiments, the bioactive agent can be covalently bound to the biodegradable stabilizing polymers via a wide variety of suitable functional groups. For example, when the biodegradable polymer is a polyester, the carboxyl group chain end can be used to react with a complimentary moiety on the bioactive agent, such as hydroxy, amino, thio, and the like. A wide variety of suitable reagents and reaction conditions are disclosed, e.g., in March's Advanced Organic Chemistry, Reactions, Mechanisms, and Structure, Fifth Edition, (2001); and Comprehensive Organic Transformations, Second Edition, Larock (1999).

In other embodiments, a bioactive agent can be linked to the PEA, PEUR or PEU polymers through an amide, ester, ether, amino, ketone, thioether, sulfinyl, sulfonyl, disulfide linkage. Such a linkage can be formed from suitably functionalized starting materials using synthetic procedures that are known in the art.

For example, in one embodiment the bioactive agent can be linked to the stabilizing polymer via an end amide or pendent carboxyl group (e.g., COOH) of the polymer. A compound of structures IV, VI, and VIII can react with an amino functional group or a hydroxyl functional group of a bioactive agent to provide a biodegradable stabilizing polymer having the bioactive agent attached via an amide linkage or carboxylic ester linkage, respectively. In another embodiment, the carboxyl group of the polymer can be benzylated or transformed into an acyl halide, acyl anhydride/“mixed” anhydride, or active ester. In other embodiments, the free —NH₂ ends of the polymer molecule can be acylated to assure that the bioactive agent will attach only via a carboxyl group of the polymer and not to the free ends of the polymer.

Affinity moieties, such as antibodies, antigens and ligands, can also be conjugated to the polymer molecules so as to be exposed on the surface lipid bilayer of the liposomal particles to target delivery of the liposomal particles to a specific body site as is known in the art and described more completely below.

A linear polymer-polypeptide conjugate is made by protecting the potential nucleophiles on the polypeptide backbone and leaving only one reactive group to be bound to the polymer or polymer linker construct. Deprotection is performed according to methods well known in the art for deprotection of peptides (Boc and Fmoc chemistry, for example).

In one embodiment of the present invention, a polypeptide bioactive agent is presented as retro-inverso or partial retro-inverso peptide.

The number of bioactive agents that can be linked to the polymer molecule can typically depend upon the molecular weight of the polymer. For example, for a compound of structural formula (I), wherein n is about 5 to about 150, preferably about 5 to about 70, up to about 150 bioactive agent molecules (i.e., residues thereof) can be directly linked to the polymer (i.e., residue thereof) by reacting the bioactive agent with side groups of the polymer. In unsaturated polymers, the bioactive agents can also be reacted with double (or triple) bonds in the polymer.

Bioactive agents suitable for use in the invention liposomal particle compositions include the anti-proliferants, rapamycin and any of its analogs or derivatives, paclitaxel or any of its taxene analogs or derivatives, everolimus, Sirolimus, tacrolimus, or any of its -limus named family of drugs, and statins such as simvastatin, atorvastatin, fluvastatin, pravastatin, lovastatin, rosuvastatin, geldanamycins, such as 17AAG (17-allylamino-17-demethoxygeldanamycin); Epothilone D and other epothilones, 17-dimethylaminoethylamino-17-demethoxy-geldanamycin and other polyketide inhibitors of heat shock protein 90 (Hsp90), Cilostazol, and the like.

Additional bioactive agents (e.g., small molecule drugs) suitable for use in the invention liposomal particle compositions include antimicrobials and anti-inflammatory agents as well as certain healing promoters, such as, for example, vitamin A and synthetic inhibitors of lipid peroxidation.

A variety of antibiotics can be used in the invention liposomal particle compositions to indirectly promote natural healing processes by preventing or controlling infection. Suitable antibiotics include many classes, such as aminoglycoside antibiotics or quinolones or beta-lactams, such as cefalosporins, e.g., ciprofloxacin, gentamycin, tobramycin, erythromycin, vancomycin, oxacillin, cloxacillin, methicillin, lincomycin, ampicillin, and colistin. Suitable antibiotics have been described in the literature.

Suitable antimicrobials include, for example, Adriamycin PFS/RDF® (Pharmacia and Upjohn), Blenoxane® (Bristol-Myers Squibb Oncology/Immunology), Cerubidine® (Bedford), Cosmegen® (Merck), DaunoXome® (NeXstar), Doxil® (Sequus), Doxorubicin Hydrochloride® (Astra), Idamycin® PFS (Pharmacia and Upjohn), Mithracin® (Bayer), Mitamycin® (Bristol-Myers Squibb Oncology/Immunology), Nipen® (SuperGen), Novantrone® (Immunex) and Rubex® (Bristol-Myers Squibb Oncology/Immunology). In one embodiment, the peptide can be a glycopeptide. “Glycopeptide” refers to oligopeptide (e.g. heptapeptide) antibiotics, characterized by a multi-ring peptide core optionally substituted with saccharide groups, such as vancomycin.

Examples of glycopeptides included in this category of antimicrobials may be found in “Glycopeptides Classification, Occurrence, and Discovery,” by Raymond C. Rao and Louise W. Crandall, (“Bioactive agents and the Pharmaceutical Sciences” Volume 63, edited by Ramakrishnan Nagarajan, published by Marcal Dekker, Inc.). Additional examples of glycopeptides are disclosed in U.S. Pat. Nos. 4,639,433; 4,643,987; 4,497,802; 4,698,327, 5,591,714; 5,840,684; and 5,843,889; in EP 0 802 199; EP 0 801 075; EP 0 667 353; WO 97/28812; WO 97/38702; WO 98/52589; WO 98/52592; and in J. Amer. Chem. Soc. (1996) 118:13107-13108; J. Amer. Chem. Soc. (1997) 119:12041-12047; and J. Amer. Chem. Soc. (1994) 116:4573-4590. Representative glycopeptides include those identified as A477, A35512, A40926, A41030, A42867, A47934, A80407, A82846, A83850, A84575, AB-65, Actaplanin, Actinoidin, Ardacin, Avoparcin, Azureomycin, Balhimyein, Chloroorientiein, Chloropolysporin, Decaplanin, -demethylvancomycin, Eremomycin, Galacardin, Helvecardin, Izupeptin, Kibdelin, LL-AM374, Mannopeptin, MM45289, MM47756, MM47761, MM49721, MM47766, MM55260, MM55266, MM55270, MM56597, MM56598, OA-7653, Orenticin, Parvodicin, Ristocetin, Ristomycin, Synmonicin, Teicoplanin, UK-68597, UD-69542, UK-72051, Vancomycin, and the like. The term “glycopeptide” or “glycopeptide antibiotic” as used herein is also intended to include the general class of glycopeptides disclosed above on which the sugar moiety is absent, i.e. the aglycone series of glycopeptides. For example, removal of the disaccharide moiety appended to the phenol on vancomycin by mild hydrolysis gives vancomycin aglycone. Also included within the scope of the term “glycopeptide antibiotics” are synthetic derivatives of the general class of glycopeptides disclosed above, included alkylated and acylated derivatives. Additionally, within the scope of this term are glycopeptides that have been further appended with additional saccharide residues, especially aminoglycosides, in a manner similar to vancosamine.

The term “lipidated glycopeptide” refers specifically to those glycopeptide antibiotics that have been synthetically modified to contain a lipid substituent. As used herein, the term “lipid substituent” refers to any substituent contains 5 or more carbon atoms, preferably, 10 to 40 carbon atoms. The lipid substituent may optionally contain from 1 to 6 heteroatoms selected from halo, oxygen, nitrogen, sulfur, and phosphorous. Lipidated glycopeptide antibiotics are well known in the art. See, for example, in U.S. Pat. Nos. 5,840,684, 5,843,889, 5,916,873, 5,919,756, 5,952,310, 5,977,062, 5,977,063, EP 667, 353, WO 98/52589, WO 99/56760, WO 00/04044, WO 00/39156, the disclosures of which are incorporated herein by reference in their entirety.

Anti-inflammatory bioactive agents are also useful for inclusion in the invention compositions and methods. Depending on the target delivery site and disease to be treated, such anti-inflammatory bioactive agents include, e.g. analgesics (e.g., NSAIDS and salicyclates), steroids, antirheumatic agents, gastrointestinal agents, gout preparations, hormones (glucocorticoids), nasal preparations, ophthalmic preparations, otic preparations (e.g., antibiotic and steroid combinations), respiratory agents, and skin & mucous membrane agents. See, Physician's Desk Reference, 2006 Edition. Specifically, the anti-inflammatory agent can include dexamethasone, which is chemically designated as (11θ, 16I)-9-fluoro-11,17,21-trihydroxy-16-methylpregna-1,4-diene-3,20-dione. Alternatively, the anti-inflammatory bioactive agent can be or include sirolimus (rapamycin), which is a triene macrolide antibiotic isolated from Streptomyces hygroscopicus.

The bioactive agent, or macromolecular biologic, conjugated to the stabilizing polymer in the invention polymer-stabilized liposomal composition can also be a peptidic antigen or protein containing antigenic segments to elicit an immune response against a wide variety of pathogens, including mucosally transmitted pathogens. The composition affords a vigorous immune response, even when the antigen is by itself weakly immunogenic. Although the vaccine compositions produced by the invention methods are broadly applicable for providing an immune response against any of the herein-described pathogens, the invention is exemplified herein by reference to influenza virus and HIV.

Peptidic antigens that generate cell-mediated immunity, and/or humoral antibody responses are a category of bioactive agents suitable for encapsulation in the invention liposomal particle compositions. Accordingly, the methods of the present invention will find use with any antigen for which cellular and/or humoral immune responses are desired, including antigens derived from viral, bacterial, fungal and parasitic pathogens that may induce antibodies, T-helper cell activity and T-cell cytotoxic activity. Thus, “immune response” as used herein means production of antibodies, T-helper cell activity or T-cell cytotoxic activity specific to the peptidic antigen used. Such antigens include, but are not limited to those encoded by human and animal pathogens and can correspond to either structural or non-structural proteins, polysaccharide-peptide conjugates, or DNA.

For example, the present invention will find use for stimulating an immune response against a wide variety of proteins from the herpes virus family, including proteins derived from herpes simplex virus (HSV) types 1 and 2, such as HSV-1 and HSV-2 glycoproteins gB, gD and gH; antigens derived from varicella zoster virus (VZV), Epstein-Barr virus (EBV) and cytomegalovirus (CMV) including CMV gB and gH; and antigens derived from other human herpes viruses such as HHV6 and HHV7. (See, e.g. Chee et al., Cytomegaloviruses (J. K. McDougall, ed., Springer-Verlag 1990) pp. 125-169, for a review of the protein coding content of cytomegalovirus; McGeoch et al., J. Gen. Virol. (1988) 69:1531-1574, for a discussion of the various HSV-1 encoded proteins; U.S. Pat. No. 5,171,568 for a discussion of HSV-1 and HSV-2 gB and gD proteins and the genes encoding therefor; Baer et al., Nature (1984) 310:207-211, for the identification of protein coding sequences in an EBV genome; and Davison and Scott, J. Gen. Virol. (1986) 67:1759-1816, for a review of VZV.)

Antigens from the hepatitis family of viruses, including hepatitis A virus (HAV), hepatitis B virus (HBV), hepatitis C virus (HCV), the delta hepatitis virus (HDV), hepatitis E virus (HEV) and hepatitis G virus (HGV), can also be conveniently used in the techniques described herein. By way of example, the viral genomic sequence of HCV is known, as are methods for obtaining the sequence. See, e.g., International Publication Nos. WO 89/04669; WO 90/11089; and WO 90/14436. The HCV genome encodes several viral proteins, including E1 (also known as E) and E2 (also known as E2/NSI) and an N-terminal nucleocapsid protein (termed “core”) (see, Houghton et al., Hepatology (1991) 14:381-388, for a discussion of HCV proteins, including E1 and E2). Each of these proteins, as well as antigenic fragments thereof, will find use in the present methods. Similarly, the sequence for the δ-antigen from HDV is known (see, e.g., U.S. Pat. No. 5,378,814) and this antigen can also be conveniently used in the present methods. Additionally, antigens derived from HBV, such as the core antigen, the surface antigen, sAg, as well as the presurface sequences, pre-S1 and pre-S2 (formerly called pre-S), as well as combinations of the above, such as sAg/pre-S1, sAg/pre-S2, sAg/pre-S1/pre-S2, and pre-S1/pre-S2, will find use herein. See, e.g., “HBV Vaccines—from the laboratory to license: a case study” in Mackett, M. and Williamson, J. D., Human Vaccines and Vaccination, pp. 159-176, for a discussion of HBV structure; and U.S. Pat. Nos. 4,722,840, 5,098,704, 5,324,513, incorporated herein by reference in their entireties; Beames et al., J. Virol. (1995) 69:6833-6838, Birnbaum et al., J. Virol. (1990) 64:3319-3330; and Zhou et al., J. Virol. (1991) 65:5457-5464.

Antigens derived from other viruses will also find use in the claimed compositions and methods, such as without limitation, proteins from members of the families Picornaviridae (e.g., polioviruses, etc.); Caliciviridae; Togaviridae (e.g., rubella virus, dengue virus, etc.); Flaviviridae; Coronaviridae; Reoviridae; Birnaviridae; Rhabodoviridae (e.g., rabies virus, etc.); Filoviridae; Paramyxoviridae (e.g., mumps virus, measles virus, respiratory syncytial virus, etc.); Orthomyxoviridae (e.g., influenza virus types A, B and C, etc.); Bunyaviddae; Arenaviridae; Retroviradae (e.g., HTLV-I; HTLV-II; HIV-1 (also known as HTLV-III LAV, ARV, hTLR, etc.)), including but not limited to antigens from the isolates HIV_{IIIb}, HIV_{SF2}, HIV_{LAV}, HIV_{LAI}, HIV_{MN}); HIV-1_{CM235}, HIV-1_{US4}; HIV-2; simian immunodeficiency virus (SIV) among others. Additionally, antigens may also be derived from human papillomavirus (HPV) and the tick-borne encephalitis viruses. See, e.g. Virology, 3rd Edition (W. K. Joklik ed. 1988); Fundamental Virology, 2nd Edition (B. N. Fields and D. M. Knipe, eds. 1991), for a description of these and other viruses.

More particularly, the envelope proteins from any of the above HIV isolates, including members of the various genetic subtypes of HIV, are known and reported (see, e.g., Myers et al., Los Alamos Database, Los Alamos National Laboratory, Los Alamos, N.Mex. (1992); Myers et al., Human Retroviruses and Aids, 1990, Los Alamos, N.Mex.: Los Alamos National Laboratory; and Modrow et al., J. Virol. (1987) 61:570-578, for a comparison of the envelope sequences of a variety of HIV isolates) and antigens derived from any of these isolates will find use in the present methods. Specifically, the synthetic peptide, R15K (Nehete et al. Antiviral Res. (2002) 56:233-251), derived from the V3 loop of gp120 and having the sequence RIQRGPGRAFVTIGK (SEQ ID NO: 1), will have use in the invention compositions and methods. Furthermore, the invention is equally applicable to other immunogenic proteins derived from any of the various HIV isolates, including any of the various envelope proteins such as gp160 and gp41, gag antigens such as p24gag and p55gag, as well as proteins derived from the pol region. Furthermore, multi-epitope cocktails of the invention composition carrying various epitopes from HIV proteins are envisioned. For example, 6 conserved peptides from gp120 and gp41 have been shown to reduce viral load and prevent transmission in a rhesus/SHIV model: SVITQACSKVSFE (S13E) (SEQ ID NO:2), GTGPCTNVSTVQC (G13C) (SEQ ID NO:3), LWDQSLKPCVKLT (L13T) (SEQ ID NO:4), VYYGVPVWKEA (V11A) (SEQ ID NO:5), YLRDQQLLGIWG (V12G) (SEQ ID NO:6), and FLGFLGAAGSTMGAASLTLTVQARQ (F25Q) (SEQ ID NO:7) (Nehete et al. Vaccine (2001) 20:813-). The amino acid sequence of the antigen tested in the invention compositions and methods is IFPGKRTIVAGQRGR (SEQ ID NO:8), wherein all amino acids are natural, L-amino acids.

As explained above, influenza virus is another example of a virus for which the present invention will be particularly useful. Specifically, the envelope glycoproteins HA and NA of influenza A are of particular interest for generating an immune response, as are the nuclear proteins. Numerous HA subtypes of influenza A have been identified (Kawaoka et al., Virology (1990) 12:759-767; Webster et al., “Antigenic variation among type A influenza viruses,” p. 127-168. In: P. Palese and D. W. Kingsbury (ed.), Genetics of influenza viruses. Springer-Verlag, New York). Thus, proteins derived from any of these isolates can also be used in the immunization techniques described herein. In particular, the conserved 13 amino acid sequence of HA can be used in the invention vaccine composition and methods. In H3 strains used in current vaccine formulations, this amino acid sequence is PRYVKQNTLKLAT (SEQ ID NO:9), and in H5 strains it is predominantly PKYVKSNRLVLAT (SEQ ID NO:10).

The compositions and methods described herein will also find use with numerous bacterial antigens, such as those derived from organisms that cause diphtheria, cholera, tuberculosis, tetanus, pertussis, meningitis, and other pathogenic organism, including, without limitation, Meningococcus A, B and C, Hemophilus influenza type B (HIB), and Helicobacter pylori. Examples of parasitic antigens include those derived from organisms causing malaria and Lyme disease.

Furthermore, the methods described herein provide a means for treating a variety of malignant cancers. For example, the composition of the present invention can be used to mount both humoral and cell-mediated immune responses to particular proteins specific to the cancer in question, such as an activated oncogene, a fetal antigen, or an activation marker. Such tumor antigens include any of the various MAGEs (melanoma associated antigen E), including MAGE 1, 2, 3, 4, etc. (Boon, T. Scientific American (March 1993):82-89); any of the various tyrosinases; MART 1 (melanoma antigen recognized by T cells), mutant ras; mutant p53; p97 melanoma antigen; CEA (carcinoembryonic antigen), among others. Additional melanoma peptidic antigens useful in the invention compositions and compositions include the following:

| | ANTIGEN | |
|---|---|---|
| DESIGNATION | SEQUENCE | PROTEIN |
| Mart1-27 | AAGIGILTV | MART1 |
| | (SEQ ID NO:11) | |
| Gp100-209* | ITDQVPFSV | Melanocyte lineage- |
| | (SEQ ID NO:12) | specific antigen GP100 |
| Gp100-154 | KTWGQYWQV | Melanocyte lineage- |
| | (SEQ ID NO:13) | specific antigen GP100 |
| Gp100-280 | YLEPGPVTA | Melanocyte lineage- |
| | (SEQ ID NO:14) | specific antigen GP100 |

*GP100 is also called melanoma-associated ME20 antigen.

To aid in stimulating an immune response, any type of adjuvant known in the art that can augment immune responses, especially cellular immune responses, to the peptidic antigen by increasing delivery of antigen, stimulating cytokine production, and/or stimulating antigen presenting cells can also be incorporated in the invention liposomal particle compositions. The adjuvants can be entrapped in the aqueous or lipid phase of the liposomal particles (depending upon the electronic properties of the adjuvants) or conjugated to the stabilizing polymer, as described herein. Such adjuvants include, but are not limited to: (1) aluminum salts (alum), such as aluminum hydroxide, aluminum phosphate, aluminum sulfate, etc.; (2) oil-in-water emulsion formulations (with or without other specific immunostimulating agents such as muramyl peptides or bacterial cell wall components), such as for example (a) MF59 (International Publication No. WO 90/14837), containing 5% Squalene, 0.5% Tween 80™, and 0.5% Span 85, optionally containing various amounts of MTP-PB, formulated into submicron particles using a microfluidizer such as Model 110Y microfluidizer (Microfluidics, Newton, Mass.), (b) SAF, containing 10% Squalane, 0.4% Tween 80™, 5% pluronic-blocked polymer L121, and thr-MDP, either microfluidized into a submicron emulsion or vortexed to generate a larger particle size emulsion, and (c) Ribi™ adjuvant composition (RAS), (Ribi Immunochem, Hamilton, Mont.) containing 2% Squalene, 0.2% Tween 80™, and one or more bacterial cell wall components from the group consisting of monophosphorylipid A (MPL), trehalose dimycolate (TDM), and cell wall skeleton (CWS), preferably MPL+CWS (Detox™); (3) saponin adjuvants, such as Stimulon™ (Cambridge Bioscience, Worcester, Mass.) may be used or particle generated therefrom such as ISCOMs (immunostimulating complexes); (4) Complete Freunds Adjuvant (CFA) and Incomplete Freunds Adjuvant (IFA); (5) cytokines, such as interleukins (IL-1, IL-2 etc.), macrophage colony stimulating factor (M-CSF), tumor necrosis factor (TNF), etc.; (6) detoxified mutants of a bacterial ADP-ribosylating toxin such as a cholera toxin (CT), a pertussis toxin (PT), or an E. coli heat-labile toxin (LT), particularly LT-K63 (where lysine is substituted for the wild-type amino acid at position 63) LT-R72 (where arginine is substituted for the wild-type amino acid at position 72), CT-S109 (where serine is substituted for the wild-type amino acid at position 109), and PT-K9/G129 (where lysine is substituted for the wild-type amino acid at position 9 and glycine substituted at position 129) (see, e.g., International Publication Nos. WO93/13202 and WO92/19265); and (7) QS21, a purified form of saponin and 3D-monophosphoryl lipid A (MPL), a nontoxic derivative of lipopolysaccharide (LPS), to enhance cellular and humoral immune responses (Moore, et al., Vaccine. 1999 Jun. 4; 17(20-21):2517-27).

Particularly desirable immunostimulating adjuvants are immunostimulating drugs (i.e., small molecules), polymers, lipids, lipid/sugars, lipid/salts, sugars, salts and biologics, examples of which are arranged by type in Table 1 below. Among these are Toll-like receptor (TLR) agonists.

TLR agonists are certain ligands, many synthetic, that are recognized by particular members of the TLR family to activate immune responses by means of the defined molecular mechanism particular to the TLR. For example, TLR-3 recognizes the synthetic drug or small molecule polyl:C; TLR-4 recognizes LPS and certain variants thereof; and TLRs-7 and 8 recognize certain drugs or small molecule ligands such as imiquimod and resiquimod. Activating immune responses at this level is analogous to using the empirically derived adjuvants, but the molecular mechanisms are better defined.

TABLE 1

| Adjuvant Name | Type |
|---|---|
| Calcitrol | Drug |
| Imiquimod | Drug |
| Loxoribine | Drug |
| Poly rA: Poly rU | Drug |
| S-28463 | Drug |
| Adjumer | Polymer |
| CRL1005 | Polymer |
| PLGA, PGA & PLA | Polymer |
| Pluronic L121 | Polymer |
| PMMA | Polymer |
| PODDS | Polymer |
| SAF-1 | Polymer |
| SPT | Polymer |
| Avridine | Lipid |
| Bay R1005 | Lipid |
| DDA | Lipid |
| DHEA | Lipid |
| DMPC | Lipid |
| DMPG | Lipid |
| D-Murapalmitine | Lipid |
| DOC/Alum Complex | Lipid |
| ISCOM | Lipid |
| Iscoprep 7.0.3 | Lipid |
| Liposomal particles | Lipid |
| MF59 | Lipid |
| Montanide ISA 51 | Lipid |
| Montanide ISA 720 | Lipid |
| MPL | Lipid |
| MTP-PE | Lipid |
| MTP-PE Liposomal particles | Lipid |
| Murapalmitine | Lipid |
| Non-Ionic Surfactant Vesicles | Lipid |
| Polysorbate 80 | Lipid |
| Protein Cochleates | Lipid |
| Span 85 | Lipid |
| Stearyl Tyrosine | Lipid |
| Theramide | Lipid |
| Gerbu Adjuvant | Lipid/Sugar |
| QS-21 | Lipid/Sugar |
| Quil A | Lipid/Sugar |
| Walter Reed Liposomal particles | Lipid/Salt |
| Algal Glucan | Sugar |
| Algammulin | Sugar |
| Gamma Inulin | Sugar |
| GMDP | Sugar |
| ImmTher | Sugar |
| Murametide | Sugar |
| Pleuran | Sugar |
| Threonyl-MDP | Sugar |
| Adju-Phos | Salt |
| Alhydrogel | Salt |
| Calcium Phosphate Gel | Salt |
| Rehydragel HPA | Salt |
| Rehydragel LV | Salt |
| Cholera Holotoxin; Cholera toxin B Subunit | Biologic |
| Cholera toxin A1-subunit-ProtienA | Biologic |
| D-fragment fusion protein | |
| Cytokine-containing liposomal particles | Biologic |
| GM-CSF | Biologic |
| immunoliposomal particles Containing Antibodies | Biologic |
| to Costimulatory Molecules (DRV) | |
| Interferon-γ | Biologic |
| Interleukin-12 | Biologic |
| Interleukin-1B | Biologic |
| Interleukin-2 | Biologic |
| Interleukin-7 | Biologic |
| LT-OA (LT-Oral Adjuvant) | Biologic |
| NAGO | Biologic |
| Sclaro Peptide | Biologic |
| Sendai Proteoliposomal particles, | Biologic |
| Sendai-containing Lipid Matrices | |
| Ty Particles | Biologic |
| Freund's Complete Adjuvant | Oil |
| Freund's Incomplete Adjuvant | Oil |
| Specol | Oil |
| Squalane | Oil |
| Squalene | Oil |

The peptides, proteins and other amino acid-containing bioactive agents used in the invention compositions and methods can also include “peptide mimetics.” Such peptide analogs, referred to herein as “peptide mimetics” or “peptidomimetics,” are commonly used in the pharmaceutical industry with properties analogous to those of the template peptide (Fauchere, J. (1986) Adv. Bioactive agent Res., 15:29; Veber and Freidinger (1985) TINS p. 392; and Evans et al. (1987) J. Med. Chem., 30:1229) and are usually developed with the aid of computerized molecular modeling. Generally, peptidomimetics are structurally similar to a paradigm polypeptide (i.e., a polypeptide that has a biochemical property or pharmacological activity), but have one or more peptide linkages optionally replaced by a linkage selected from the group consisting of: —CH₂NH—, —CH₂S—, CH₂—CH₂—, —CH═CH—(cis and trans), —COCH₂—, —CH(OH)CH₂—, and —CH₂SO—, by methods known in the art and further described in the following references: Spatola, A. F. in “Chemistry and Biochemistry of Amino Acids, Peptides, and Proteins,” B. Weinstein, eds., Marcel Dekker, New York, p. 267 (1983); Spatola, A. F., Vega Data (March 1983), Vol. 1, Issue 3, “Peptide Backbone Modifications” (general review); Morley, J. S., Trends. Pharm. Sci., (1980) pp. 463-468 (general review); Hudson, D. et al., Int. J. Pept. Prot. Res., (1979) 14:177-185 (—CH₂NH—, CH₂CH₂—); Spatola, A. F. et al., Life Sci., (1986) 38:1243-1249 (—CH₂—S—); Harm, M. M., J. Chem. Soc. Perkin Trans I (1982) 307-314 (—CH═CH—, cis and trans); Almquist, R. G. et al., J. Med. Chem., (1980) 23:2533 (—COCH₂—); Jennings-Whie, C. et al., Tetrahedron Lett., (1982) 23:2533 (—COCH₂—); Szelke, M. et al., European Appln., EP 45665 (1982) CA: 97:39405 (1982) (—CH(OH)CH₂—); Holladay, M. W. et al., Tetrahedron Lett., (1983) 24:4401-4404 (—C(OH)CH₂—); and Hruby, V. J., Life Sci., (1982) 31:189-199 (—CH₂—S—). Such peptide mimetics may have significant advantages over natural polypeptide embodiments, including, for example: more economical production, greater chemical stability, enhanced pharmacological properties (half-life, absorption, potency, efficacy, etc.), altered specificity (e.g., a broad-spectrum of biological activities), reduced antigenicity, and others.

Additionally, substitution of one or more amino acids within a peptide (e.g., with a D-Lysine in place of L-Lysine) may be used to generate more stable peptides and peptides resistant to endogenous peptidases. Alternatively, the synthetic polypeptides covalently bound to the biodegradable polymer, can also be prepared from D-amino acids, referred to as inverso peptides. When a peptide is assembled in the opposite direction of the native peptide sequence, it is referred to as a retro peptide. In general, polypeptides prepared from D-amino acids are very stable to enzymatic hydrolysis. Many cases have been reported of preserved biological activities for retro-inverso or partial retro-inverso polypeptides (U.S. Pat. No. 6,261,569 B1 and references therein; B. Fromme et al. Endocrinology (2003)144:3262-3269.

### The Affinity Moiety

The affinity moiety, as will be described below, is a peptide or polymer ligand effective to bind specifically and with high affinity to ligand-binding molecules carried on a target, such as cell membrane, a cell matrix, a tissue or target surface or region at which the liposomal particle-based delivery is aimed. The affinity moiety is bound to the outer liposomal particle surface by covalent attachment to the stabilizing polymer molecules or to ligands conjugated to the stabilizing polymer as described herein. For example, an affinity moiety can be conjugated to a distal end of a PEA, PEUR or PEUR lipophilic polymer chain or to a free functional group in the polymer molecule.

In one embodiment, the affinity moiety is effective to bind to a tumor-specific antigen in a solid tumor and, in another embodiment, the affinity moiety is effective to bind to cells at a site of inflammation. In another embodiment, the affinity moiety is a polypeptide or polysaccharide effector molecule effective to inhibit a cell-binding event, that is, to interfere with binding between a first binding member and a second binding member. For example, the first binding member can be a pathogen or cell in the bloodstream and the second binding member can be a target cell or cell matrix.

TABLE 2
LIGAND-RECEPTOR PAIRS AND ASSOCIATED TARGET CELL

| LIGAND | RECEPTOR | CELL TYPE |
|---|---|---|
| Folate | folate receptor | epithelial carcinomas |
| | | bone marrow stem cells |
| water soluble vitamins | vitamin receptor | various cells |
| pyridoxyl phosphate | CD4 | CD4 + lymphocytes |
| apolipoproteins | LDL | vascular endothelial |
| | | cells |
| insulin | insulin receptor | liver hepatocytes |
| transferrin | transferrin receptor | endothelial cells (brain) |
| galactose | asialoglycoprotein receptor | liver hepatocytes |
| sialyl-Lewis* | E, P selectin | activated endothelial |
| cells Mac-1 | L selectin | neutrophils, leukocytes |
| VEGF | Flk-1,2 | tumor epithelial cells |
| basic FGF | FGF receptor | tumor epithelial cells |
| EGF | EGF receptor | epithelial cells |
| VCAM-1 | α₄β₁ integrin | vascular endothelial |
| cells ICAM-1 | α_{L}β₂ integrin | vascular endothelial |
| cells PECAM-1/CD31 | αᵥβ₃ integrin | vascular endothelial |
| cells fibronectin | αᵥβ₃ integrin | activated platelets |
| osteopontin | αᵥβ₁ and αᵥβ₅ integrins | endothelial cells and smooth |
| | | muscle cells in atherosclerotic |
| | | plaques |
| RGD sequences of | αᵥβ₃integrin | tumor endothelial cells |
| matrix proteins | | vascular smooth muscle cells |
| HIV GP 120/41 or GP120 | CD4 | CD4 + lymphocytes |
| C4 domain peptomers | | |
| HIV/GP 120/41 (T cell | fusin | CD4 + lymphocytes |
| tropic isolates) or | | |
| SDF-1 chemokines | | |
| HIV GP 120/41 (Macro- | Chemokine receptor | macrophages, dendritic cells |
| phage tropic isolates) | CC-CRK-5 | |
| Anti-cell surface receptor | cell surface receptors | erythrocytes, platelets |
| antibodies (or fragments | | bone marrow stem cells |
| such as CD-34 thereof) | | |

The ligands in Table 2 can be conjugated to the stabilizing polymer molecules in the liposomal particles to target the liposomal particles to specific target cell type, where a bioactive agent entrapped in the liposomal particles will be delivered to the target cells. For example, a surface-bound folate ligand conjugated to a PEA stabilizing polymer molecule in the liposomal particle will bind to a folate receptor on epithelial cells to deliver an entrapped neoplastic agent for treatment of epithelial carcinomas.

Additional categories of surface-bound affinity moieties useful in the invention compositions and methods are a variety of well known polypeptide cytokines, which are effective as mediators of natural immunity, such as IFN-alpha or beta.

The preferred polymers for use in preparation of invention compositions for in vivo administration of macromolecular biologics are the PEA, PEUR and PEU polymers having chemical structures described by formulas (I and IV-VIII). Such polymers possess sufficient lipophilic properties to stabilize the three-dimensional structure of cargo biologic macromolecules via the same non-covalent forces that are found within native macromolecular biologics, and aggregates thereof. These stabilizing forces arise from discrete hydrophobic segments along the polymer chains, which give rise to short-range dispersion forces, and charged or partially charged regions of the polymer, which give rise to localized ionic interactions, including hydrogen bonds. In particular, in the invention polymer compositions for macromolecular biologics, hydrogen bonding may occur directly between polymer and macromolecular biologic, or may be bridged via a discrete water molecule in a manner equivalent to the slowly exchangeable, bound water molecules found at the surface of native biologic macromolecules and which form a bridge between macromolecules in aggregates thereof, such as crystals.

### Enhancement of Loading and Stability by Polymer-Enhanced Oligomerization or Crystallization of Macromolecular Biologics in Liposomal Particles

Due to the hydrocarbon segments contained therein, the synthetic PEAs, PEURs, and PEUs described herein are not soluble in water. However, they are partially wettable, probably because individual water molecules can hydrogen-bond to the amino acid residues, and thereby form hydrogen bonded bridges to more water molecules. It is believed that these bound water molecules are important for the stabilization of interactions between the polymer and macromolecular biologics, in much the same way as discrete, bound water molecules have been demonstrated to be essential for the stabilization of macromolecular biologic structures and of higher order structures, such as oligomers and crystals.

Crystalline arrays of biological molecules in which the crystallites are formed under mild conditions represent natural or quasi-natural configurations that can achieve optimal packing density, while stabilizing the macromolecular structure. Indeed, some proteins, e.g. pro-insulin, are naturally preserved within storage granules as micro-crystalline aggregates.

In nature, many macromolecular biologics exist as a quaternary structure, which structure often represents the active biological configuration. Examples of macromolecular biologics that exist as a quaternary structure include some nucleic acids (anti-parallel, double helical dimers), many gene-regulatory proteins (DNA-binding dimers of two protomers), the transport proteins hemoglobin and transthyretin (each a quartet of protomers), the enzyme aspartate transcarbamoylase (six regulatory plus catalytic protomers), iscosahedral virus coats (multiples of sixty protomers), helical virus coats (Tobacco Mosaic virus has 2130 protomers), and cell-structural assemblies, such as actin and tubulin cables (composed of many thousands of protomers).

In macromolecular biologics two or more such identical protein molecules or protomers can bind together non-covalently, but specifically, so as to form a protein oligomer. The spatial arrangement of the protomers is called the quaternary structure of the oligomer. In most oligomers with biological activity, the protomers are spatially related by simple rotational symmetries. However, many oligomeric proteins crystallize with more than one protomer in the crystallographic asymmetric unit, so these symmetries are not necessarily exact. An example of a quaternary configuration of protomers commonly observed in crystal structures of oligomeric proteins is that of dimers that are related by additional rotational symmetries. The resulting oligomer, which may, or may not represent the biologically active configuration, is more stable and has a lower free-energy minimum than a simple translational crystalline aggregate of the protomer. For example, human insulin readily dimerizes and, in the presence of zinc atoms, three dimers assemble around a three-fold axis of symmetry to form a stable hexamer of molecules. Under suitable conditions, these soluble hexamers can be aggregated to form crystals in which hexamer-hexamer interactions are further stabilized by zinc atoms. For macromolecular biologics other than insulin, atoms of other transition metals or calcium may facilitation aggregation of oligomers to form crystals. The art of crystallization of macromolecular biologics has progressed to the point that oligomers or crystals of virtually any polypeptide or protein can be obtained by those of skill in the art of crystallography.

The example of crystallization of insulin is described herein to illustrate an important general feature of crystallization of macromolecular biologics, such as proteins. The non-covalent electronic forces that bind the crystal are similar in type and strength to those that stabilize the quaternary structure of an oligomer, and that indeed maintain the three-dimensional folding of the protein molecule (i.e., the protomer) itself.

Thus, the three-dimensional folded structure of a macromolecular biologic can be preserved in the invention PEA, PEUR and PEU polymer particle compositions described herein by a combination of hydrophobic and ionic bonding of the macromolecular biologic: 1) to the polymer, 2) to spatially neighboring copies of the macromolecular biologic itself (i.e., micro-crystallization, with or without oligomerization), and, optionally, 3) to spatially neighboring copies of the macromolecular biologic itself (i.e., crystallization, with or without oligomerization) in which, a minority of protomers have been conjugated to the polymer As illustrated in FIG. 1 and exemplified in Example 1 herein, it is envisioned that these polymer-conjugated protomers act as seed molecules, promoting the crystallization, with or without oligomerization and under mild conditions, of surrounding free protomers, thereby stabilizing the three-dimensional structure of the protomers, and so preserving biological function of the macromolecular biologic.

Accordingly in one embodiment the invention provides liposomal particle compositions in which at least one macromolecular biologic is dispersed in the biodegradable polymer used in the invention compositions and methods, for example one comprising at least one PEA, PEUR or PEU having a chemical formula described by any one of structural formulas (I) or (III-VII). The at least one macromolecular biologic can be conjugated to the polymer prior to introduction of the polymer into the solution containing the lipids and lipid-acting compounds used to formulate the liposomal particles. Formation of a macromolecular biologic-polymer conjugate is illustrated herein in the Examples by conjugation of insulin monomer to PEA. The macromolecular biologic-containing conjugate can then be concentrated into an oligomer (e.g., an insulin hexamer with zinc) and crystallized using a dialysis method as described in Example 1 herein, and as known in the art. This procedure is completed prior to introduction of the polymer-biologic conjugate into the lipid phase used during liposomal particle formation. This procedure utilizes sufficiently mild conditions to protect the “folded” (i.e., three dimensional structure) of the macromolecular biologic in the conjugate throughout liposomal particle formation.

In still another embodiment, the invention provides methods for delivering a macromolecular biologic with substantial native activity to a subject by administering to the subject in vivo an invention polymer-stabilized liposomal composition comprising a stabilizing polymer of structural formulas (I), or (IV-VIII) and having conjugated thereto at least one macromolecular biologic monomer, which composition biodegrades by enzymatic action in vivo to release the macromolecular biologic with substantial native activity in a controlled manner.

The compositions will generally include one or more “pharmaceutically acceptable excipients or vehicles” appropriate for oral, mucosal or subcutaneous delivery, such as water, saline, glycerol, polyethylene glycol, hyaluronic acid, ethanol, and the like. Additionally, auxiliary substances, such as wetting or emulsifying agents, pH buffering substances, flavorings, and the like, may be present in such vehicles.

The invention compositions can be administered as lyophilized powders, for example, orally or by nasal administration. For example, intranasal and pulmonary formulations will usually include vehicles that neither cause irritation to the nasal mucosa nor significantly disturb ciliary function. In other embodiments, diluents such as water, aqueous saline or other known substances can be employed with the subject invention compositions and formulations. The intrapulmonary formulations may also contain preservatives such as, but not limited to, chlorobutanol and benzalkonium chloride. A surfactant may be present to enhance absorption by the nasal mucosa.

Additional, molecules and vehicles that can be used for oral delivery with favorable physical chemical properties to reduce the solid-liquid surface tension and free energy changes and facilitate permeability across the intestinal wall, but minimal or no negative physiological/toxic properties include compounds that are Generally Recognized As Safe (GRAS), listed in the FDA Guidelines for Inactive Ingredients, or have undergone the necessary toxicity and tolerability studies as defined by official pharmaceutical regulatory agencies. Categories of molecules and vehicles that have an effect on the permeability of the intestine are bile salts, non-ionic surfactants, ionic surfactants, fatty acids, glycerides, acyl carnitines, cholines, salicylates, chelating agents, and swellable polymers. Examples of these molecules and vehicles that fall in this category include, but are not limited to natural, semisynthetic, and synthetic: phospholipids, polyethylene triglycerides, gelatin, ionic surfactants (sodium lauryl sulfate), non-ionic surfactants, e.g., dioctyl sodium sulfosuccinate, Tween and Cremaphore, bile acids and bile acid derivatives, digestable oils, e.g., cottonseed, corn, soybean, and olive, citric acid, EDTA, stearoyl macrogoglycerides, lauroyl macrogoglycerides, propylene glycol derivatives, i.e., propylene glycol caprylate and monocaprylate, propylene glycol laurate and monolaurate, oleoyl macrogolglycerides, caprylocaproyl macrogolglycerides, glycerol monolinoleate, glyceryl monooleate, polyglyceryl oleate, glycerol esters of fatty acids, medium chain triglycerides, sodium caprate, acyl carnitines and cholines, salicylates, e.g., sodium salicylate and methoxysalicylate, chitosan, starch, polycarbophil, N-acetylated α-amino acids, N-acetylated non-α-amino acids, 12-hydroxy stearic acid, and diethylene glycol monoethyl ether. Competitive substrates and protease inhibitors, for example compounds such as pancreatic inhibitor, soybean trypsin inhibitor, FK-448, camostat mesylate, aprotinin, p-chloromericuribenzoate, and bacitracin are also included in this list.

Furthermore for oral delivery, coatings that help protect the liposomal particles from pH initiated degradation include, but are not limited to, shellac, cellulose acetate, cellulose acetate butyrate, cellulose acetate phthalate, methacrylic acid copolymers, e.g., polymethacrylate amino-ester copolymer, hydroypropyl methyl cellulose phthalate, ethyl cellulose, and poly vinyl acetate phthalate. A variety of enteric coatings are commercially available.

For a further discussion of appropriate vehicles to use for particular modes of delivery, see, e.g., Remington: The Science and Practice of Pharmacy, Mack Publishing Company, Easton, Pa., 19th edition, 1995. One of skill in the art can readily determine the proper vehicle to use for the particular macromolecular biologic/polymer particle combination, size of particle and mode of administration.

Any suitable and effective amount of the at least one biologic agent can be released with time from the invention composition and will typically depend, e.g., on the specific stabilizing polymer, lipid composition, and type of liposomal particle or polymer/bioactive agent conjugation, if present. Typically, up to about 100% of the bioactive agent can be released from an invention composition administered in vivo. Specifically, up to about 90%, up to 75%, up to 50%, or up to 25% thereof can be released from the composition. Factors that typically affect the release rate from the invention polymer-stabilized liposomal compositions are the nature and amount of the stabilizing polymer, the composition of the liposomal particle bilayers, the type of liposomal particles in which the composition is formulated, the types of polymer/bioactive agent linkage, and the nature and amount of additional substances present in the formulation.

The polymer-stabilized liposomal compositions can be formulated to provide a variety of properties. In one embodiment, the liposomal particles are sized to agglomerate in vivo forming a time-release polymer depot for local delivery of bioactive agents to surrounding tissue/cells when injected in vivo, for example subcutaneously, intramuscularly, or into an interior body site, such as an organ. Invention liposomal particles of sizes capable of passing through pharmaceutical syringe needles ranging in size from about 19 to about 27 Gauge, for example those having an average diameter in the range from about 1 μm to about 100 μm, can be injected into an interior body site, and will form a depot to dispense the bioactive agent(s) locally and systemically. In other embodiments, the invention composition acts as a carrier for the bioactive agent into the circulation for targeted and timed release systemically. Liposomal particles in the size range of about 10 nm to about 500 nm will enter directly into the circulation for such purposes.

The biodegradable polymers used in the invention polymer-stabilized liposomal composition can be designed to tailor the rate of biodegradation of the polymer to result in continuous delivery of the bioactive agent over a selected period of time. For instance, typically, the invention compositions, as described herein, will biodegrade over a time selected from about 15 days to about five months, depending upon the body site into which the composition is administered, although the sequestered bioactive agent may not be present in the composition throughout this time range. Longer time spans within the above range are particularly suitable for providing a composition that eliminates the need to repeatedly administer the composition to obtain a suitable therapeutic or palliative response.

Once the invention composition is made, as above, the compositions are formulated for subsequent in vivo delivery and will generally include one or more “pharmaceutically acceptable excipients or vehicles” appropriate for systemic administration, such as water, saline, glycerol, polyethylene glycol, hyaluronic acid, ethanol, etc. Additionally, auxiliary substances, such as wetting agents, pH buffering substances, and the like, may be present in such vehicles. For a further discussion of appropriate vehicles to use for particular modes of delivery, see, e.g., Remington: The Science and Practice of Pharmacy, Mack Publishing Company, Easton, Pa., 19th edition, 1995. One of skill in the art can readily determine the proper vehicle to use for the particular bioactive agent/liposomal particle combination, size of liposomal particle and mode of administration.

The compositions used in the invention methods optionally may comprise an “effective amount” of the bioactive agent(s) of interest. That is, an amount of a bioactive agent may be included in the compositions that will cause the subject to produce a sufficient therapeutic or palliative response in order to prevent, reduce or eliminate symptoms. The exact amount necessary will vary, depending on the subject being treated; the age and general condition of the subject to be treated; the severity of the condition being treated; the particular bioactive agent selected and formulation, among other factors. An appropriate effective amount can be readily determined by one of skill in the art. Thus, an “effective amount” will fall in a relatively broad range that can be determined through routine trials. For example, for purposes of the present invention, an effective amount will typically range from about 1 μg to about 100 mg, for example from about 5 μg to about 1 mg, or about 10 μg to about 500 μg of the bioactive agent delivered per dose.

Once formulated, the invention liposomal particle compositions can be administered systemically by any route known in the art, for example, by intravenous, parenteral, interperitoneal, oral, nasal, subcutaneous, rectal, or ocular delivery, for example, by intravitreal, subconjunctival, topical, subtenon or periorbital delivery to the eye. Dosage treatment may be a single dose of the invention liposomal particle composition, or a multiple dose schedule as is known in the art. The dosage regimen, at least in part, will also be determined by the need of the subject and be dependent on the judgment of the practitioner.

Dosage treatment may be a single dose of the invention liposomal particle composition, or a multiple dose schedule as is known in the art. The dosage regimen, at least in part, will also be determined by the bioactive agent entrapped in the liposomal particles, need of the subject and be dependent on the judgment of the practitioner. Furthermore, if prevention of disease is desired, the liposomal particle composition is generally administered for delivery of the bioactive agent prior to primary disease manifestation, or symptoms of the disease of interest. If treatment is desired, e.g., the reduction of symptoms or recurrences, the liposomal particle compositions is generally administered for delivery of the bioactive agent subsequent to primary disease manifestation.

The formulations can be tested in vivo in a number of animal models developed for the study of oral, subcutaneous, or mucosal delivery. Blood samples can be assayed for the macromolecular biologic using standard techniques, as known in the art.

The following examples are meant to illustrate, but not to limit the invention.

### Example 1

### Preparation of Polymer-Insulin Conjugate

Activation of PEA. The PEA (65 kD) co-polymer (386 mg, 209 μmol of CO₂H) was dissolved in DMF (1.0 mL) and stirred with N-hydroxysuccinamide (13.22 mg, 115 μmol) and DCC (23.78 mg, 115 μmol) at RT for 24 h. The reaction mixture was filtered through a frit (0.2 μm) and washed with 0.5 mL of DMF.

Conjugation (PEA-Ins). The activated ester in DMF was mixed with 1 equiv of insulin (597 mg) in DMSO (3 mL) and diisopropylethyl amine (54 μL, 3.0 equiv) and stirred for 48 h. PEA-Insulin (PEA-Ins) conjugate solution was examined by GPC and forwarded to the next step, synthesis of Core Material.

### Fabrication of Polymer-Stabilized Liposomal Particles (Oil-in-Water Method)

Step 1: Stabilization of the biologic with polymer (Core Material PEA-Ins-Ins[Hex]): All solutions and dialysis were stored at 4° C. in deaerated solutions.

Stock solution A: 650 mg zinc sulfate and 490 mg phenol were dissolved in 20 ml water to give a concentration of 32.5 mg/ml, and an equilibrated pH of 5.6.

Stock solution B: 0.5 ml of stock solution A was dissolved in 0.5 L water and the pH was adjusted to 3.7.

Stock solution C: 4 ml of stock solution A were mixed with 4 L of water and the pH was adjusted to 6.6.

Stock solution D: Insulin was dissolved in stock solution B to a concentration of 20 mg/ml and the pH was re-adjusted back to 3.7.

Then, 90 mL of insulin-containing stock solution D were added to a dialysis chamber, followed by 10 mL of the PEA-Ins protomer conjugate in DMSO/DMF. The chamber contents were dialyzed against 4 L of stock solution C. The buffer was changed twice per day and dialysis was carried out for 3 days. The material from the dialysis bag was then collected and lyophilized to give the desired product. The insulin content in the PEA core batches calculated from GPC (PS) and HPLC chromatograms ranged from 82% to 94%.

GPC measurements calibrated with polystyrene standards were conducted in Dimethylacetamide/LiCl (0.1%) using a Waters 2487 system, equipped with Refractive Index Detector (Waters 2414) and two Styragel HR 5E DMF columns (7.8×300 mm, Waters) and a Waters Styragel Guard column (4.6×30 mm). For HPLC measurements a Jupiter 5 μm C4, 300A, 250×4.6 mm column was used with the security Guard Cartridge: Millipore C4 (AVO-4330). Mobile Phase A: 80% Water+20% Acetonitrile with 0.1% TFA. Mobile Phase B: 80% Acetonitrile+20% Water with 0.1% TFA according to the following method:

| | |
|---|---|
| Time | Mobile Phase B |

| | | | |
|---|---|---|---|
| 0-3 | minutes | 20% | isocratic |
| 3-6 | minutes | 20%-100% | gradient |
| 6-11 | minutes | 100% | isocratic |
| 11-12 | minutes | 100%-20% | isocratic |
| 12-15 | minutes | 20% | isocratic |
| 15-21 | minutes | 20% | isocratic |

Step 2: emulsification. For phase A, components were dissolved in 1,1,1,3,3,3-Hexafluoro-2-propanol (HFIP) or dichloromethane DCM before addition together so as to form a homogeneous solution (phase A) as follows: (1) 1388 mg PEA-Ins-Ins[Hex] (from Step 1) in 27.76 ml HFIP, (2) 416 mg of sodium cholate in 4.16 mL HFIP, (3) 83.3 mg of cottonseed oil in 0.833 mL DCM, (4) 694.1 mg of lethicin in 6.94 mL DCM, (5) 55.5 mg of cholesterol in 0.555 mL DCM and (6) 55.5 mg of oleic acid in 0.555 mL HFIP. Once all components were a homogenous solution, phase A was then injected via a 40 mL syringe equipped with a 20 gauge needle into 500 mL of De-Ionized water (liquid B) containing dissolved poly(vinyl) alcohol (139 mg) at 4° C. during ultrasonication at 25 W of power over 60 seconds (Fisher Scientific Sonic Dismembrator, model 100).

Step 3: stabilization. The emulsion underwent roto-evaporation at ca. 11 mm Hg vacuum for 600 seconds to remove the organic solvent, then was frozen in liquid nitrogen, and finally was lyophilized overnight. Recovered yield ranged in 87±7%. The range in yield was likely the result of removal of larger particles prior to lyophilization of final product. Stabilized particles were analyzed by dynamic light scattering (Malvern ZS 90), TLC, GPC and HPLC technique. The formulations had an average diameter as measured by intensity of 196.7±3.6 nm and a mean zeta potential of −43.8±2.7 mV (in DI water at a concentration 1 mg/mL, at 23° C.). By thin layer chromatography (TLC) with mobile phase (v/v) Chloroform (72%), Methanol (25%), H₂O (3%), the following compound R_{f}s were identified: soy lethicin (0.12), sodium cholate (0.28), cholesterol (0.80) and cotton seed oil triglycerides (0.94). Anisaldehyde stain used to visualize cottonseed oil, oleic acid, cholesterol, cholic acid, and lethicin: Mix of 18.4 mL p-anisaldehyde, 3.75 mL Acetic acid, 338 mL 95% Ethanol, and 12.5 mL Sulfuric acid. Estimated insulin (hexamer) content in formulation was measured as 431±26 μg/mg by HPLC and 454±38 μg/mg by GPC.

Step 4 (optional): re-constitution in vitro. For in vitro testing, the final product was re-constituted from a dry powder in a variety of aqueous media, including De-Ionized water, hydroxypropyl methyl cellulose, PBS, and mannitol solutions.

### Insulin Release

The release of insulin from the formulations, which were designed to be quick release, was tested in vitro in buffers of neutral and acidic pH to simulate exposure to the conditions in the intestine and stomach, respectively. Briefly, a known amount of polymer stabilized liposomal particles formulation was suspended in either PBS pH 7.4 or 10 mM HCl pH 1.8 at a normalized insulin concentration of 0.5 mg/mL. The particle suspension was stirred at 4° C. for 5 minutes and then centrifuged at 13,000 rcf for 10 minutes at 4° C. Both the supernatant and pellet were analyzed for insulin content by HPLC. Measurements showed that at 4° C. in 5 min time period 84±3.2% Insulin was released in pH 7.4 PBS buffer and 92±4% was released in 10 mM HCl solution at pH 1.8.

### Preparation of Lipid Phase for Liposomal Formulation

The lipid and/or lipid-acting molecules used in preparation of the lipid phase from Class (I-IV), were as follows:

Class I, oleic acid and cottonseed oil;

Class II, lecithin-phosphatidylcholine;

Class III, poly (vinyl alcohol);

Class IV, cholesterol, and sodium cholate.

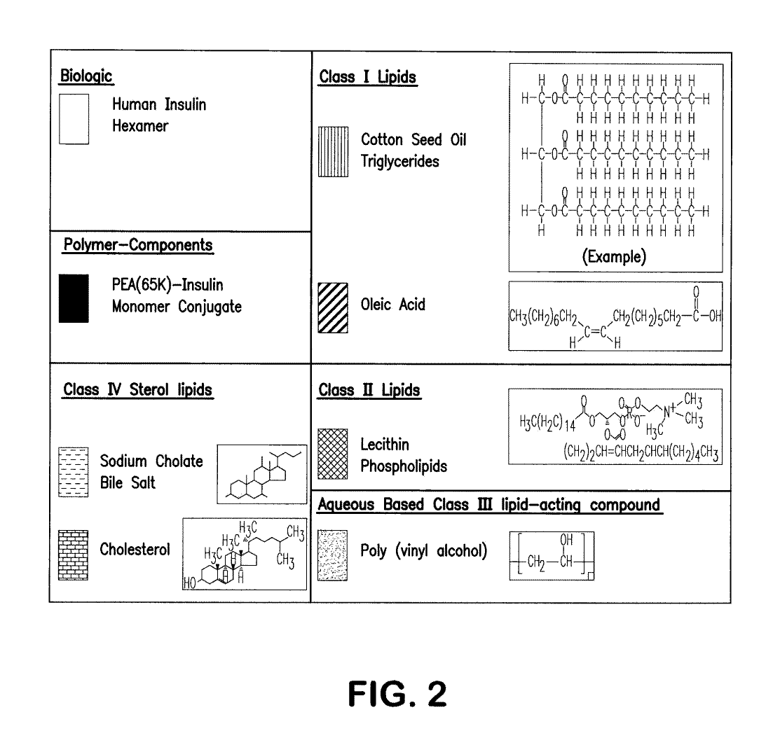

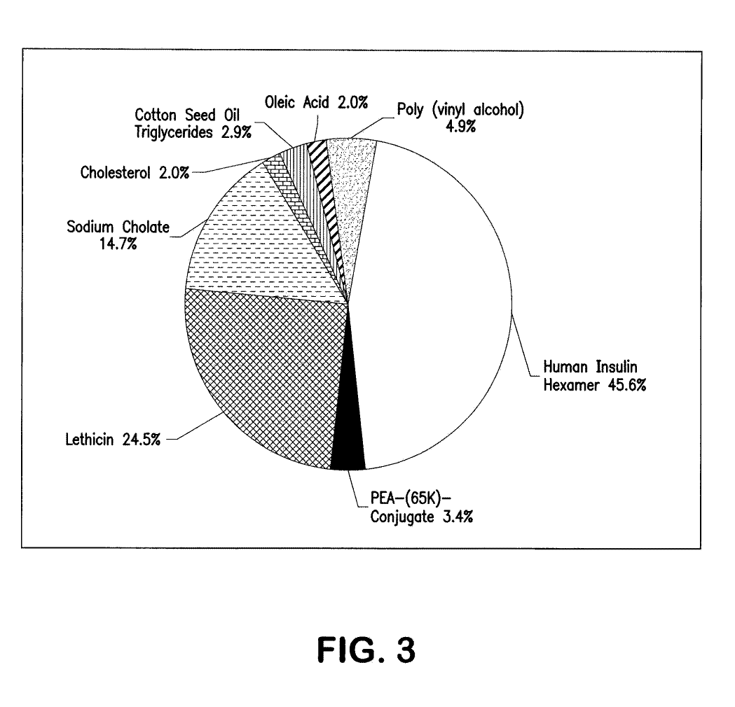

The various components of the polymer-stabilized liposomal particles are shown schematically in FIG. 2. The calculated mean proportions (wt %) of these components per polymer stabilized liposomal particle is shown in FIG. 3, using the same shading scheme as in FIG. 2.

Only natural molecules that moreover are components of food were used in this example because the composition is intended for delivery of insulin through the wall of the small intestine and thereby into portal vein blood. The clinical indications for use of this type of oral formulation are Type-I and Type-2 diabetes.

### Example 2

### Bioactivity of Insulin Delivered Orally in a Pea-Stabilized Liposomal Formulation Encapsulated in Gelatin Capsules Covered with an Enteric Coating

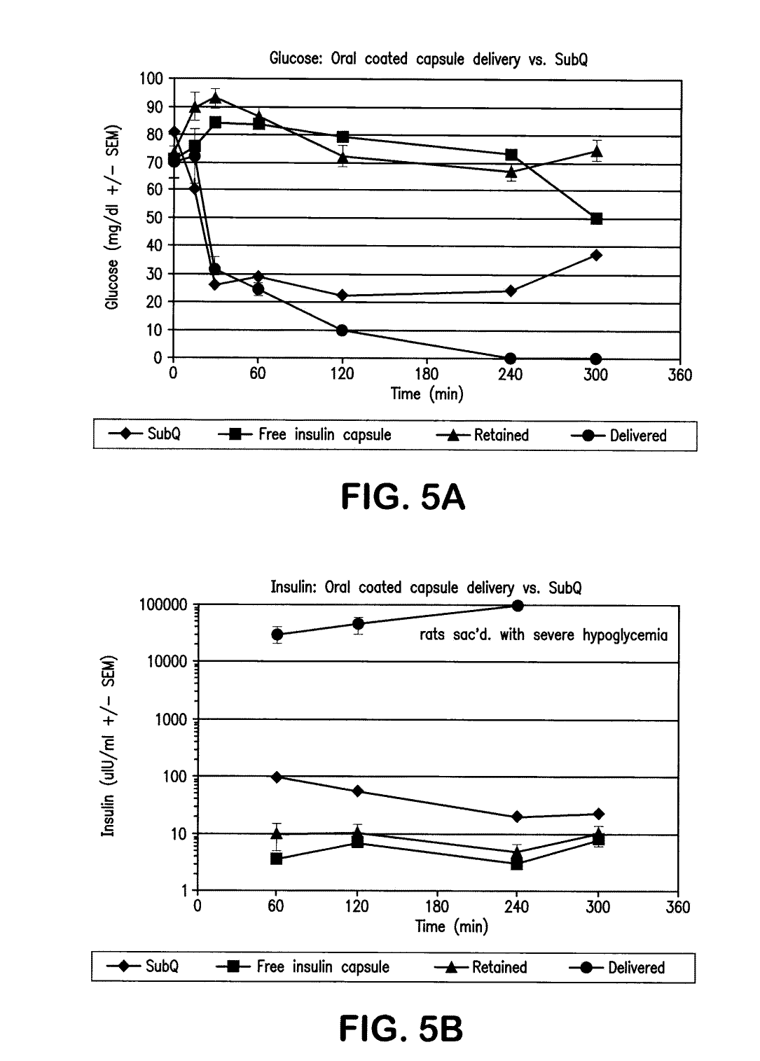

PEA-insulin formulation, made as described in Example 1 above, was encapsulated in gelatin capsules that were then encased within an enteric coating. The capsules were administered to fasted rats such that each rat received a dose of 60 IU/kg insulin. At the time points shown in FIGS. 5A and B, blood was collected to measure blood glucose and insulin levels compared to a non-polymer-encapsulated insulin dose of 1 IU/kg injected subcutaneously (subQ).

A separate study using contrast agent-filled capsules showed that the capsules do not always exit the stomach within a 5 hour time period. Therefore, based on the results of the contrast agent study, the results shown in FIGS. 5A and B are separately designated as “Delivered” capsules, if glucose lowering and blood insulin were measured, meaning the capsules ultimately delivered the insulin payload into the bloodstream. When the insulin payload could not be measurably detected in the blood, capsules were designated “Retained”, indicating that the capsules may not have exited the stomach over the course of the study.

A significant and sustained lowering of levels of glucose and of insulin were measured in rats receiving the invention PEA-stabilized insulin liposomal formulation in their small intestine, such that the rats were euthanized to prevent suffering from severe hypoglycemia. Over the 3 hour period beginning 1 hour post administration, the estimated insulin delivered was 20× (2,000%) than was delivered by the 1 IU/Kg Sub Q dose, as measured by area under the respective curves shown in FIGS. 5A and 5B. Furthermore, delivery of insulin using enteric coated capsules results in no additional bioavailability or bioactivity of insulin systemically.

### Bioactivity of Insulin Delivery Orally in PEA Particles

PEA-insulin formulations prepared as in Example 1 above were suspended in phosphate-buffered saline to deliver doses of 10, 30 and 60 IU/kg to fasted rats. The solutions were administered by oral gavage, and a non-polymer-encapsulated insulin dose of 1 IU/kg was injected subcutaneously (subQ) as a positive control.

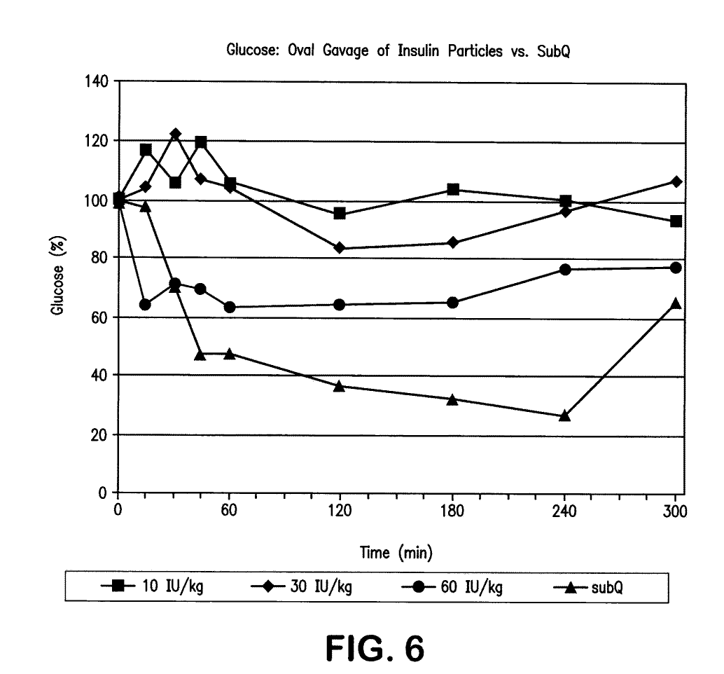

The measurements of glucose levels were taken over approximately three hours. The results were calculated as a percentage of the glucose level at time zero for each dose (FIG. 6). A 40% decrease in blood glucose level over approximately 3 hours resulted by oral delivery of a dose of 60 IU of insulin/kg of body weight. A 20% decrease in blood glucose level over about 2 hours resulted from oral delivery of a dose of 30 IU/kg, and no decrease was measured at a dose of 10 IU of insulin/kg of rat body weight. Therefore, this example shows that insulin was delivered orally from the invention PEA-stabilized liposomal composition that was subsequently absorbed through the intestines and was bioactive in reducing blood glucose levels.

### Example 3

### Fabrication of Polymer-Stabilized Liposomal Particles by Oil-in-Oil Method

Lecithin (25.8 mg), cholic acid (15.5 mg), cholesterol (2.1 mg), capmul C10 (3.1 mg), and oleic acid (2.1 mg) was dissolved in 0.5 mL of methanol. This solution was then mixed with PEA-Ins-Ins[Hex] (53.1 mg) dissolved in 1.0 mL HFIP to form a discontinuous phase. A continuous phase was made by mixing 0.4% Span 80 into 80 mL of cottonseed oil. The discontinuous phase was premixed with 12% by volume of the continuous phase to yield a pre-emulsion. This pre-emulsion was added to the continuous phase and emulsified at room temperature for 15 min at 6000 rpm. The organic solvents were removed by rotoevaporation. The material was collected on a 0.8 μm nylon filter. After lyophilization the average mass yield of 6 batches was 61±2 mg of material obtained as a white powder to give a 61±2% yield (wt/wt). The insulin loading in the particles (wt/wt) was determined to be 67±4% by HPLC and 63±3% by GPC. The DLS analysis gave a bimodal peak distribution with peak 1=136.2 nm (51.1%) and peak 2=525.6 nm (45.9%). The amount of insulin released in 5 min in PBS, pH=7.4 at 4° C. was 84%±3.2%. The amount of insulin released in 5 min in PBS, pH=1.8 at 4° C. was 91.2%±4.3%. TLC analysis detected the presence of PEA-Ins-Ins[Hex], lecithin, cholic acid and triglycerides.

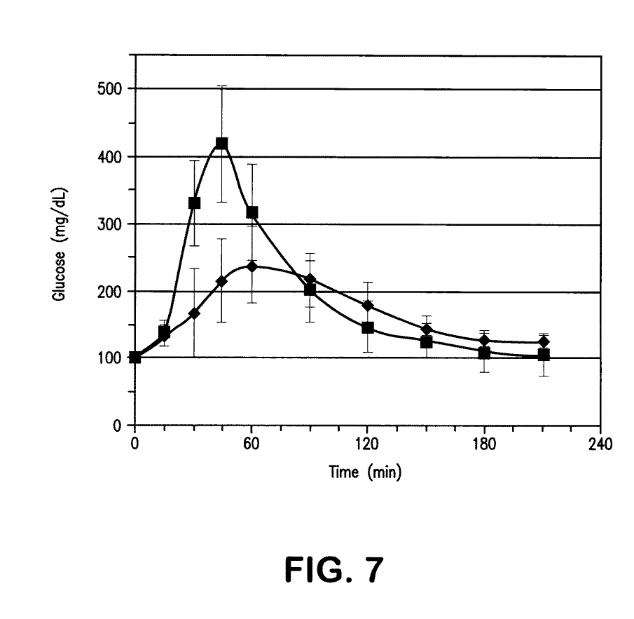

Bioactivity of orally delivered polymer-stabilized liposomal particles made using an oil-in-oil method was tested as described (FIG. 7). Following an overnight fast, groups of mice (n=6) were given an oral gavage with and without a formulation containing 10 IU/kg insulin. At 15 min post dosing, the mice were given an oral glucose challenge of 2.5 g/kg. In the no formulation (▪) control mice, glucose levels increased significantly to a maximum of approximately 450 mg/dL at 45 minutes. However, in the mice that received the formulation (♦), the glucose levels reached only 250 mg/dL due to the presence of human insulin delivered by the formulation.

### Example 4

### Fabrication of Polymer-Stabilized Liposomal Particles with Enteric Coating

Stock solutions of 10% Cholic Acid in Methanol, 10% Eudragit L100 in MeOH and 5% PEA-Ins-Ins[Hex] in HFIP were made. The continuous phase was made by mixing of 0.5% Span 80 into 80 mL cottonseed oil. The discontinuous phase was prepared by mixing 1.0 mL of PEA-Ins-Ins[Hex] with 300 μL of cholic acid stock solution while sonicating and then 1.2 mL Eudragit L100 was added. Discontinuous phase was added to the continuous phase and emulsified at room temperature for 15 min at 8000 rpm. The volatile organic solvents were removed by rotoevaporation and then diluted with 80 mL of hexanes and stored at 4° C. overnight. Particles were collected on a 0.8 μm nylon filter and rinsed with hexanes. After lyophilization, the average mass yield of 2 batches was 152±3 mg of material obtained as a white powder to give a 76±1% yield (wt/wt). The insulin loading in the particles (wt/wt) was determined to be 22% by HPLC.

Formulation degradation studies in Pepsin (pH 1.8) at 37° C. after 15 min showed that 107±5% of the undegraded insulin was present in the particles (HPLC). The DLS analysis gave a unimodal peak distribution with 389±94 nm (>90%) diameter. The amount of insulin released in 5 min in PBS, pH=7.4 at 4° C. was 100%. The amount of insulin released in 5 min in PBS, pH=2.0 at 4° C. was 0%.

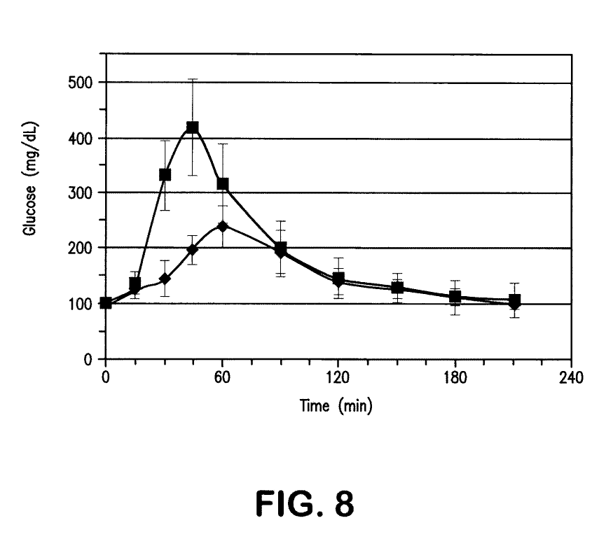

Bioactivity of orally delivered polymer-stabilized liposomal particles coated with enteric coating were tested as described (FIG. 8): Following an overnight fast, groups of mice (n=6) were given an oral gavage with (♦) and without (▪) formulation at 10 IU/kg insulin. At 15-minutes post dosing, the mice were given an oral glucose challenge of 2.5 g/kg. In the no formulation control mice, glucose levels increased significantly to a maximum of approximately 450 mg/dL at 45 minutes post dosing. However, in the mice that received the invention composition, the glucose levels reached only 230 mg/dL due to the presence of human insulin delivered by the composition.

Although certain of the individual components of the invention polymer-stabilized liposomal particle compositions described herein were known, it was unexpected and surprising that such combinations of components would enhance the efficiency of time release systemic delivery of the incorporated bioactive agents beyond levels achieved when the components were used separately.

All publications, patents, and patent documents are incorporated by reference herein, as though individually incorporated by reference. The invention has been described with reference to various specific and preferred embodiments and techniques. However, it should be understood that many variations and modifications might be made while remaining within the spirit and scope of the invention.

Although the invention has been described with reference to the above examples, it will be understood that modifications and variations are encompassed within the spirit and scope of the invention. Accordingly, the invention is limited only by the following claims.

What is claimed is:

1. A polymer-stabilized liposomal composition comprising:
a) a liposomal particle comprising:
a stabilizing polymer with lipophilic properties, which polymer is integrally located throughout the liposomal particle to provide structural support thereto; and
at least one vesicle-forming lipid or lipid-acting compound; and
b) at least one bioactive agent entrapped in concert by the stabilizing polymer and the lipid or lipid-acting compound so as to retain substantial native activity thereof.

2. The composition of claim 1, wherein the composition is lyophilized.

3. The composition of claim 1, further comprising an enteric coating encapsulating the liposomal particles.

4. The composition of claim 1, wherein the stabilizing polymer comprises at least one or a blend of the following polymers:
a PEA having a chemical formula described by structural formula (I), 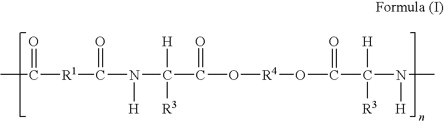
wherein n ranges from about 5 to about 150; R¹ is independently selected from the group consisting of (C₂-C₂₀) alkylene, (C₂-C₂₀) alkenylene, α,ω-bis(4-carboxyphenoxy) (C₁-C₈) alkane, residues of 3,3′-(alkanedioyldioxy) dicinnamic acid or 4,4′-(alkanedioyldioxy) dicinnamic acid, residues of α,ω-alkylene dicarboxylates of formula (III), and combinations thereof; wherein R⁵ and R⁶ in formula (III) are independently (C₂-C₁₂) alkylene or (C₂-C₁₂) alkenylene; the R³s in individual n monomers are independently selected from the group consisting of hydrogen, (C₁-C₆) alkyl, (C₂-C₆) alkenyl, (C₆-C₁₀) aryl (C₁-C₆) alkyl and —(CH₂)₂SCH₃; and R⁴ is independently selected from the group consisting of (C₂-C₂₀) alkylene, (C₂-C₂₀) alkenylene, (C₂-C₈) alkyloxy (C₂-C₂₀) alkylene, bicyclic-fragments of 1,4:3,6-dianhydrohexitols of structural formula (II), and combinations thereof; 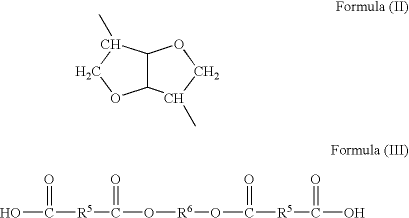
or a PEA having a chemical formula described by structural formula (IV): 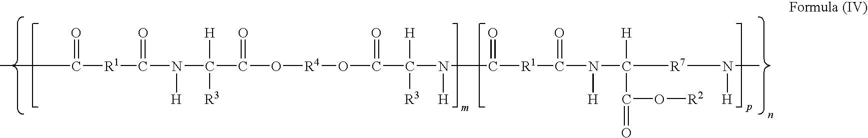
wherein n ranges from about 5 to about 150, m ranges about 0.1 to 0.9: p ranges from about 0.9 to 0.1; wherein R¹ is independently selected from the group consisting of (C₂-C₂₀) alkylene, (C₂-C₂₀) alkenylene, α,ω-bis(4-carboxyphenoxy) (C₁-C₈) alkane, residues of 3,3′-(alkanedioyldioxy) dicinnamic acid or 4,4′-(alkanedioyldioxy) dicinnamic acid, residues of α,ω-alkylene dicarboxylates of formula (III); wherein R⁵ and R⁶ in Formula (III) are independently (C₂-C₁₂) alkylene or (C₂-C₁₂) alkenylene; each R² is independently selected from the group consisting of hydrogen, (C₁-C₁₂) alkyl, (C₂-C₈) alkyloxy (C₂-C₂₀) alkyl, (C₆-C₁₀) aryl and a protecting group; the R₃s in individual m monomers are independently selected from the group consisting of hydrogen, (C₁-C₆) alkyl, (C₂-C₆) alkenyl, (C₆-C₁₀) aryl (C₁-C₆) alkyl and —(CH₂)₂SCH₃; and R⁴ is independently selected from the group consisting of (C₂-C₂₀) alkylene, (C₂-C₂₀) alkenylene, (C₂-C₈) alkyloxy (C₂-C₂₀) alkylene, bicyclic-fragments of 1,4:3,6-dianhydrohexitols of structural formula (II), and combinations thereof, and R⁷ is independently (C₁-C₂₀) alkyl or (C₂-C₂₀) alkenyl;
or a poly(ester urethane) (PEUR) having a chemical formula described by structural formula (V), 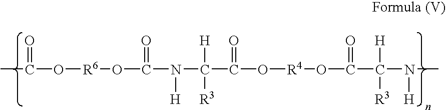
and wherein n ranges from about 5 to about 150; wherein the R³s within an individual n monomer are independently selected from the group consisting of hydrogen, (C₁-C₆) alkyl, (C₂-C₆) alkenyl, (C₆-C₁₀) aryl(C₁-C₆) alkyl and —(CH₂)₂SCH₃; R⁴ and R⁶ are selected from the group consisting of (C₂-C₂₀) alkylene, (C₂-C₂₀) alkenylene, (C₂-C₈) alkyloxy (C₂-C₂₀) alkyl, selected from the group consisting of bicyclic-fragments of 1,4:3,6-dianhydrohexitols of structural formula (II), and combinations thereof;
or a PEUR having a chemical structure described by general structural formula (VI), 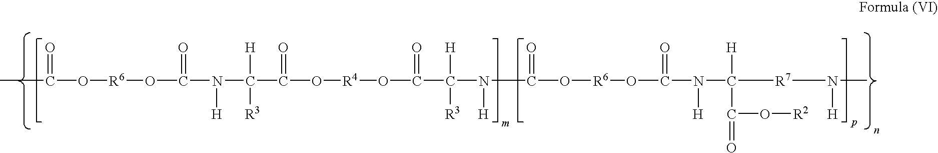
wherein n ranges from about 5 to about 150, in ranges about 0.1 to about 0.9: p ranges from about 0.9 to about 0.1; R² is independently selected from the group consisting of hydrogen, (C₁-C₁₂) alkyl, (C₂-C₈) alkyloxy (C₂-C₂₀) alkyl, (C₆-C₁₀) aryl and a protecting group; the R³s within an individual m monomer are independently selected from the group consisting of hydrogen, (C₁-C₆) alkyl, (C₂-C₆) alkenyl, (C₆-C₁₀) aryl (C₁-C₆) alkyl and —(CH₂)₂SCH₃; R⁴ and R⁶ are independently selected from (C₂-C₂₀) alkylene, (C₂-C₂₀) alkenylene, (C₂-C₈) alkyloxy (C₂-C₂₀) alkyl, bicyclic-fragments of 1,4:3,6-dianhydrohexitols of structural formula (II), and combinations thereof, and R⁷ is independently (C₁-C₂₀) alkyl or (C₂-C₂₀) alkenyl;
or a poly(ester urea) (PEU) having a chemical formula described by structural formula (VII), 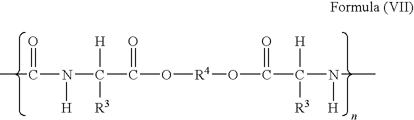
wherein n is about 10 to about 150; the R³s within an individual n monomer are independently selected from the group consisting of hydrogen, (C₁-C₆) alkyl, (C₂-C₆) alkenyl, (C₆-C₁₀) aryl (C₁-C₆)alkyl and —(CH₂)₂SCH₃; R⁴ is independently selected from (C₂-C₂₀) alkylene, (C₂-C₂₀) alkenylene, (C₂-C₈) alkyloxy (C₂-C₂₀) alkylene; and a bicyclic-fragment of a 1,4:3,6-dianhydrohexitol of structural formula (II) and combinations thereof;
or a PEU having a chemical formula described by structural formula (VIII), 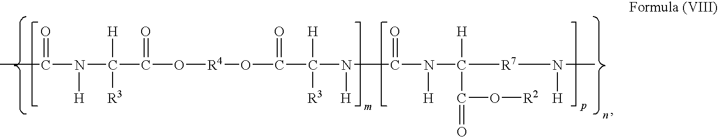
wherein m is about 0.1 to about 1.0; p is about 0.9 to about 0.1; n is about 10 to about 150; R² is independently selected from the group consisting of hydrogen, (C₁-C₁₂) alkyl, (C₂-C₈) alkyloxy (C₂-C₂₀) alkyl, (C₆-C₁₀) aryl and a protecting group; the R³s within an individual m monomer are independently selected from the group consisting of hydrogen, (C₁-C₆) alkyl, (C₂-C₆) alkenyl, (C₆-C₁₀) aryl (C₁-C₆)alkyl and —(CH₂)₂SCH₃; R⁴ is independently selected from the group consisting of (C₂-C₂₀) alkylene, (C₂-C₂₀) alkenylene, (C₂-C₈) alkyloxy (C₂-C₂₀) alkylene; or a bicyclic-fragment of a 1,4:3,6-dianhydrohexitol of structural formula (II), and a combination thereof, and R⁷ is independently (C₁-C₂₀) alkyl or (C₂-C₂₀) alkenyl.

5. The composition of claim 4, wherein the composition further comprises discrete water molecules hydrogen bonded to the stabilizing polymer and to the bioactive agent.

6. The composition of claim 4, wherein the stabilizing polymer has a chemical structure described by structural formula (IV), (VI) or (VIII) and the bioactive agent is covalently attached thereto via an amide or carboxyl group of the polymer.

7. The composition of claim 1, wherein the composition biodegrades over a period of about 5 to 12 hours to about 8 hours to 5 months.

8. The composition of claim 1, wherein the liposomal particle has an average diameter in the size range from about 100 μm to about 10 nm.

9. The composition of claim 4, wherein the at least one bioactive agent comprises a biologic that is conjugated to a functional group in the stabilizing polymer.

10. The composition of claim 9, wherein the biologic is a monomeric peptide, protein, or oligonucleotide.

11. The composition of claim 4, wherein the composition further comprises a Class III, soluble amphiphilic surfactant and/or coating and the lipid or lipid-acting molecule is at least one or a combination of:
Class I, non-polar and non-swelling stabilizers;
Class II, polar but insoluble, swelling amphiphiles; and
Class IV, cholesterol or cholesterol based compounds.

12. The composition of claim 4, wherein the biologic comprises a macromolecular biologic monomer conjugated to the biodegradable stabilizing polymer so as to maintain substantial native activity thereof.

13. The composition of claim 12, wherein the macromolecular biologic is an insulin monomer.

14. The composition of claim 12, wherein the macromolecular biologic monomer is incorporated into a protein aggregate or crystal thereof, which comprises additional unattached monomers of the macromolecular biologic.

15. The composition of claim 4, wherein concentration of the biologic in the product composition is in the range from about 5% to about 60% by weight.

16. The composition of claim 1, wherein the bioactive agent is hydrophilic.

17. The composition of claim 1, wherein the bioactive agent is a hydrophobic drug.

18. The composition of claim 1, wherein the bioactive agent is a peptidic antigen and the composition further comprises an immunostimulating adjuvant entrapped therein.

19. A method for delivering a bioactive agent with substantial native activity to a subject comprising administering to the subject in vivo a composition of claim 1, which composition biodegrades to release the bioactive agent in vivo to the subject with the substantial native activity at a controlled rate.

20. The method of claim 19, wherein the administering is by a route selected from the group consisting of intravenous, parenteral, interperitoneal, oral, nasal, subcutaneous, rectal, and ocular routes.

21. The method of claim 19, wherein the composition is administered as a lyophilized powder.

22. A method for making a polymer-stabilized liposomal composition, said method comprising:
a) combining the following components in a first liquid to form a first homogeneous liquid solution:
i) at least one bioactive agent having a native activity,
ii) a stabilizing polymer with lipophilic properties, and
iii) at least one lipid or lipid-acting compound selected from the group consisting of
Class I, non-swelling and non-polar stabilizers;
Class II, polar but insoluble, swelling amphiphiles; and
Class IV, cholesterol or cholesterol based compounds; and
b) emulsifying the first homogeneous liquid solution in a second liquid in which the components are not soluble to obtain an emulsion of droplets of the first homogeneous liquid solution in the second liquid; and
c) evaporating the first liquid from the emulsion of droplets so as to yield stable liposomal particles comprising the components i), ii) and iii) with the bioactive agent entrapped in concert by the stabilizing polymer and the at least one lipid or lipid-acting compound so as to retain substantial native activity.

23. The method of claim 22, wherein the stabilizing polymer is at least one or a blend of polymers whose chemical structures are described by structural formulas (I and IV-VIII).

24. The method of claim 23, wherein the at least one bioactive agent comprises a biologic.

25. The method of claim 22, wherein the at least one bioactive agent comprises an insulin monomer and the method further comprises using the insulin monomer conjugated to the polymer as a seed to form an oligomer thereof in the presence of excess insulin monomers.


SEQUENCE LISTING (14 sequences)

SEQ ID NO: 1 - protein, 15 aa, Human immunodeficiency virus
RIQRGPGRAFVTIGK

SEQ ID NO: 2 - protein, 13 aa, Human immunodeficiency virus
SVITQACSKVSFE

SEQ ID NO: 3 - protein, 13 aa, Human immunodeficiency virus
GTGPCTNVSTVQC

SEQ ID NO: 4 - protein, 13 aa, Human immunodeficiency virus
LWDQSLKPCVKLT

SEQ ID NO: 5 - protein, 11 aa, Human immunodeficiency virus
VYYGVPVWKEA

SEQ ID NO: 6 - protein, 12 aa, Human immunodeficiency virus
YLRDQQLLGIWG

SEQ ID NO: 7 - protein, 25 aa, Human immunodeficiency virus
FLGFLGAAGSTMGAASLTLTVQARQ

SEQ ID NO: 8 - protein, 15 aa, Human immunodeficiency virus
IFPGKRTIVAGQRGR

SEQ ID NO: 9 - protein, 13 aa, Influenza virus
PRYVKQNTLKLAT

SEQ ID NO: 10 - protein, 13 aa, Influenza virus
PKYVKSNRLVLAT

SEQ ID NO: 11 - protein, 9 aa, Artificial sequence
  Synthetic construct
AAGIGILTV

SEQ ID NO: 12 - protein, 9 aa, Artificial sequence
  Synthetic construct
ITDQVPFSV

SEQ ID NO: 13 - protein, 9 aa, Artificial sequence
  Synthetic construct
KTWGQYWQV

SEQ ID NO: 14 - protein, 9 aa, Artificial sequence
  Synthetic construct
YLEPGPVTA